(19) United States
(12) Patent Application Publication
(10) Pub. No.: US 2020/0143931 A1
(43) Pub. Date: May 7, 2020

(54) ELECTRONIC DEVICES AND METHODS FOR TREATMENT UTILIZING ANTIPSYCHOTICS IN COMBINATION WITH DIGITAL THERAPIES

(71) Applicants: Novartis AG, Basel (CH); Pear Therapeutics, Inc., Boston, MA (US)

(72) Inventors: Seyyed Nassir GHAEMI, Cambridge, MA (US); Aaron Justin BEAUDETTE, Oakland, CA (US); Timothy Ryan CAMPELLONE, Oakland, CA (US); Justin KODAMA, Alameda, CA (US)

(21) Appl. No.: 16/448,907

(22) Filed: Jun. 21, 2019

Related U.S. Application Data
(60) Provisional application No. 62/757,077, filed on Nov. 7, 2018.

(51) Int. Cl. G16H 20/70; A61B 5/16; G16H 10/20

(52) U.S. Cl. CPC G16H 20/70; A61M 2021/0044; G16H 10/20; A61B 5/165

(57) ABSTRACT

Electronic devices and methods are provided for the treatment of schizophrenia utilizing delivery of an antipsychotic in combination with computerized behavioral therapy.

### CROSS-REFERENCE TO RELATED APPLICATIONS

This application claims priority to U.S. Provisional Patent Application Ser. No. 62/757,077, entitled “ELECTRONIC DEVICES AND METHODS FOR TREATMENT UTILIZING ANTIPSYCHOTICS IN COMBINATION WITH DIGITAL THERAPIES,” filed Nov. 7, 2018, the contents of which is hereby incorporated by reference in its entirety.

### FIELD

This disclosure relates, generally, to the treatment of disorders (e.g., schizophrenia) and, more particularly, to electronic devices and methods for the treatment of such disorders utilizing delivery of one or more antipsychotics in combination with computerized behavioral therapy.

### BACKGROUND

Schizophrenia is a common condition occurring in about 1% of the general population. It is treated with antipsychotic medications, which can help acute exacerbations of delusions or hallucinations, but do not improve the long-term course of the illness.

Cognitive-behavioral therapy (CBT) has been shown to improve symptoms of schizophrenia; however, access to CBT is frequently not readily available. CBT traditionally is administered by a psychotherapist. Accordingly, access to CBT has typically been restricted due to, among other factors, lack of availability of psychotherapists and cost.

Unfortunately, these obstacles to treatment often leave patients vulnerable, particularly during periods when the symptoms associated with schizophrenia are acute.

Accordingly, what is needed are electronic devices and methods for addressing one or more of the drawbacks associated with traditional treatment of schizophrenia.

### SUMMARY

This disclosure relates to:

i) The use of one or more antipsychotics in combination with a computer-implemented mobile device:
in the treatment of schizophrenia;
in the treatment of positive symptoms of schizophrenia;
in the treatment of negative symptoms of schizophrenia;
in the treatment of depression associated with schizophrenia;
in the treatment of insomnia associated with schizophrenia;
   wherein the computer-implemented mobile device includes:
a display;
an input device;
one or more processors;
memory storing one or more programs configured to be executed by the one or more processors, the one or more programs including instructions for:
   displaying, on the display, a query interface, the query interface presenting a plurality of queries, including a first query;
   while displaying the query interface, receiving, via the input device, a first sequence of inputs;
   in response to receiving the first sequence of inputs, displaying a response summary interface, the response summary interface including:
      in accordance with the response to the first query being a response of a first type, displaying, in a first region of the response summary interface, a first response element corresponding to the first query, the first response element including at least a first selectable graphical element corresponding to a first set of digital therapies associated with the first query; and
      in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

ii) The use of an antipsychotic and a digital-therapeutic:
in the treatment of schizophrenia;
in the treatment of positive symptoms of schizophrenia;
in the treatment of negative symptoms of schizophrenia;
in the treatment of depression associated with schizophrenia;
in the treatment of insomnia associated with schizophrenia;
   wherein the digital-therapeutic delivers behavioral therapy, such as cognitive behavioral therapy for schizophrenia.

iii) The use of an antipsychotic:
in the treatment of schizophrenia;
in the treatment of positive symptoms of schizophrenia;
in the treatment of negative symptoms of schizophrenia;
in the treatment of depression associated with schizophrenia;
in the treatment of insomnia associated with schizophrenia;
   wherein the use is combined with computer-implemented behavioral therapy, such as computer-implemented cognitive behavioral therapy for schizophrenia.

### BRIEF DESCRIPTION OF DRAWINGS

For a better understanding of the various described examples, reference should be made to the detailed description, below, in conjunction with the following drawings in which like reference numerals refer to corresponding parts throughout the figures.

FIG. 1 is a block diagram illustrating a portable multifunction device with a touch-sensitive display in accordance with some embodiments.

FIGS. 2A-2J illustrate exemplary user interfaces for targeted delivery of digital therapies, in accordance with some examples.

FIGS. 3A-3I illustrate exemplary user interfaces for notifications associated with a mobile application for providing targeted delivery of digital therapies, in accordance with some examples.

FIG. 4 illustrates an exemplary state diagram showing the relationships and progressions between various user interfaces for targeted delivery of digital therapies and/or for notifications associated with a mobile application for providing targeted delivery of digital therapies, in accordance with some examples.

FIGS. 5A-5B are flow diagrams illustrating a method for targeted delivery of digital therapies, in accordance with some examples.

### DETAILED DESCRIPTION

The combination of an antipsychotic and a computer-implemented behavioral therapy, such as a computer-implemented cognitive behavioral therapy in accordance with the embodiments set forth herein, may prove effective for treating schizophrenic patients. Among other advantages, the electronic devices and methods described herein may provide the following improvements over conventional systems and techniques for treating schizophrenia:
i) alleviating (e.g., by eliminating or by reducing intensity), for example compared to a sham or placebo, one or more of “symptoms associated with schizophrenia” [e.g., items according to the PANSS scale], for example as assessed (e.g., by % improvement in score relative to baseline) from the total PANSS score;
ii) alleviating (e.g., by eliminating or by reducing intensity,), for example compared to a sham or placebo, one or more of “positive symptoms associated with schizophrenia” [i.e., items P1 to P7 of the Positive Scale (P), according to the PANSS scale], for example as assessed (e.g., by % improvement in score relative to baseline) from the Positive PANSS score, or as assessed (e.g., by % improvement in score relative to baseline) from the Positive and General Psychopathology PANSS scores;
iii) alleviating (e.g., by eliminating or by reducing intensity,), for example compared to a sham or placebo, one or more of “negative symptoms associated with schizophrenia” [i.e., items N1 to N7 of the Negative Scale (N), according to the PANSS scale], for example as assessed (e.g., by % improvement in score relative to baseline) from the Negative PANSS score, or as assessed (e.g., by % improvement in score relative to baseline) from the Negative PANSS score and Motivation and Pleasure Scale-Self Report (MAP-SR) score;
iv) alleviating (e.g., by eliminating or by reducing intensity,), for example compared to a sham or placebo, depressive symptoms associated with schizophrenia, for example as assessed from the Beck Depression Inventory Second Edition (BDI-II) score [e.g., Beck, A.T., Brown, G. K. (1996), Manual for the Beck Depression Inventory-II, San Antonio, Tex.: Psychological Corporation]
v) alleviating (e.g., by eliminating or by reducing intensity,), for example compared to a sham or placebo, insomnia associated with schizophrenia, for example as assessed from the Insomnia Severity Index (ISI) score [ e.g., Sleep, 2011; 34(5):601-608];
vi) increasing retention of patients in treatment, for example, compared to a sham or placebo, for example it increases the rate of patient retention in a treatment program, such as a clinical setting (e.g., as measured by patient attendance at scheduled clinic visits, time to dropout from treatment or % dropout rate);
vii) decreasing relapse of schizophrenia symptoms, for example, compared to a sham or placebo, for example it increases the time to relapse; viii) improving medication adherence, for example, antipsychotic medication adherence, for example compared to a sham or placebo, for example as assessed by Brief Medication Questionnaire [BMQ; e.g., in Patient Educ Couns., 1999, 37(2), 113-124];
ix) improving psychosocial functioning, for example as assessed by the World Health Organization Quality of Life (WHOQOL-BREF) scale score [e.g., WHOQOL-BREF, Introduction, administration, Scoring and Generic Version of the Assessment, Field Trial Version, December 1996, World Health Organization; http://www.who.int/mental health/media/en/76.pdf; Psychol Med 1998, 28(3), 551-558], for example compared to a sham or placebo:
x) providing a favorable therapeutic profile, for example compared to a sham or placebo, such as a favorable safety profile, for example a favorable safety profile in relation to psychiatric adverse events [e.g., no increase of depression or suicidality e.g., as assessed by the InterSePT Scale for Suicidal Thinking-Plus (ISST-Plus) score [e.g., Innov Clin Neurosci., 2014, 11(9-10), 32-46]].

Accordingly, therapeutic advantages of a combination in accordance with the embodiments set forth herein comprise one or more of i) to x) listed above.

Embodiments of the present invention are:

### Embodiments (a)

1a. A combination for use in the treatment of schizophrenia comprising an antipsychotic and a computer-implemented mobile device, wherein the computer-implemented mobile device comprises:
a display;
an input device;
one or more processors; and
memory storing one or more programs configured to be executed by the one or more processors, the one or more programs including instructions for:
   displaying, on the display, a query interface, the query interface presenting a plurality of queries, including a first query;
   while displaying the query interface, receiving, via the input device, a first sequence of inputs;
   in response to receiving the first sequence of inputs, displaying a response summary interface, the response summary interface including:
      in accordance with the response to the first query being a response of a first type, displaying, in a first region of the response summary interface, a first response element corresponding to the first query, the first response element including at least a first selectable graphical element corresponding to a first set of digital therapies associated with the first query; and
      in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

2a. A combination for use in the treatment of positive symptoms of schizophrenia comprising an antipsychotic and a mobile device, wherein the computer-implemented mobile device comprises:
a display;
an input device;
one or more processors; and
memory storing one or more programs configured to be executed by the one or more processors, the one or more programs including instructions for:
   displaying, on the display, a query interface, the query interface presenting a plurality of queries, including a first query;
   while displaying the query interface, receiving, via the input device, a first sequence of inputs;
   in response to receiving the first sequence of inputs, displaying a response summary interface, the response summary interface including:
      in accordance with the response to the first query being a response of a first type, displaying, in a first region of the response summary interface, a first response element corresponding to the first query, the first response element including at least a first selectable graphical element corresponding to a first set of digital therapies associated with the first query; and
      in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

3a. The combination for use according to embodiment 2a, wherein positive symptoms of schizophrenia are one or more selected from the group comprising delusions associated with schizophrenia, disorganized thinking associated with schizophrenia, hallucinations associated with schizophrenia, excitement associated with schizophrenia, grandiosity associated with schizophrenia, suspiciousness associated with schizophrenia and hostility associated with schizophrenia.

4a. A combination for use in the treatment of negative symptoms of schizophrenia comprising an antipsychotic and a computer-implemented mobile device, wherein the computer-implemented mobile device comprises:
a display;
an input device;
one or more processors; and
memory storing one or more programs configured to be executed by the one or more processors, the one or more programs including instructions for:
   displaying, on the display, a query interface, the query interface presenting a plurality of queries, including a first query;
   while displaying the query interface, receiving, via the input device, a first sequence of inputs;
   in response to receiving the first sequence of inputs, displaying a response summary interface, the response summary interface including:
      in accordance with the response to the first query being a response of a first type, displaying, in a first region of the response summary interface, a first response element corresponding to the first query, the first response element including at least a first selectable graphical element corresponding to a first set of digital therapies associated with the first query; and
      in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

5a. The combination for use according to embodiment 4a, wherein negative symptoms of schizophrenia are one or selected from the group comprising blunted affect associated with schizophrenia, emotional withdrawal associated with schizophrenia, poor rapport associated with schizophrenia, apathetic social withdrawal associated with schizophrenia, difficulty in abstract thinking associated with schizophrenia, lack of spontaneity and flow of conversation associated with schizophrenia and stereotyped thinking associated with schizophrenia.

6a. A combination for use in the treatment of depression associated with schizophrenia comprising an antipsychotic and a computer-implemented mobile device, wherein the computer-implemented mobile device comprises:
a display;
an input device;
one or more processors; and
memory storing one or more programs configured to be executed by the one or more processors, the one or more programs including instructions for:
   displaying, on the display, a query interface, the query interface presenting a plurality of queries, including a first query;
   while displaying the query interface, receiving, via the input device, a first sequence of inputs;
   in response to receiving the first sequence of inputs, displaying a response summary interface, the response summary interface including:
      in accordance with the response to the first query being a response of a first type, displaying, in a first region of the response summary interface, a first response element corresponding to the first query, the first response element including at least a first selectable graphical element corresponding to a first set of digital therapies associated with the first query; and
      in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

7a. A combination for use in the treatment of insomnia associated with schizophrenia comprising an antipsychotic and a computer-implemented mobile device, wherein the computer-implemented mobile device comprises:
a display;
an input device;
one or more processors; and
memory storing one or more programs configured to be executed by the one or more processors, the one or more programs including instructions for:
   displaying, on the display, a query interface, the query interface presenting a plurality of queries, including a first query;
   while displaying the query interface, receiving, via the input device, a first sequence of inputs;
   in response to receiving the first sequence of inputs, displaying a response summary interface, the response summary interface including:
      in accordance with the response to the first query being a response of a first type, displaying, in a first region of the response summary interface, a first response element corresponding to the first query, the first response element including at least a first selectable graphical element corresponding to a first set of digital therapies associated with the first query; and
      in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

8a. The combination for use according to any one of embodiments 1a to 7a, wherein the computer-implemented mobile device is system-prompted or on-demand.

9a. The combination for use according to any one of embodiments 1a to 8a, wherein the computer-implemented mobile device is selected from the group comprising a smartphone, a laptop computer, a tablet computer and a wearable computer.

10a. The combination for use according to any one of embodiments 1a to 9a, which comprises one or more active agent (i.e., at least one further active agent).

11a. The combination for use according to embodiment 10a, wherein the antipsychotic and the one or more active agent (i.e., at least one further active agent) is in the form of a fixed pharmaceutical combination or a non-fixed pharmaceutical combination.

12a. The combination for use according to embodiments 10a or 11a, wherein the further active agent is selected from the group comprising an antidepressant and an anxiolytic.

13a. The combination for use according to any one of embodiments 1a to 11a, wherein the computer-implemented mobile device delivers behavioral therapy.

14a. The combination for use according to embodiment 13a, wherein the behavioral therapy is cognitive behavioral therapy for schizophrenia, such as cognitive behavioral therapy for schizophrenia that focuses on symptoms of psychosis, mood management, sleep hygiene, medication adherence or social skills.

15a. The combination for use according to embodiment 13a, wherein the behavioral therapy is cognitive behavioral therapy for schizophrenia, such as cognitive behavioral therapy for schizophrenia that focuses on one or more of (i.e., on at least one of) i) to v): i) symptoms of psychosis, ii) mood management, iii) sleep hygiene, iv) medication adherence and v) social skills.

16a. The combination for use according to any one of embodiments 1a to 15a, wherein the first query is associated with a first symptom of schizophrenia and the first set of digital therapies is configured to address the first symptom of schizophrenia.

17a. The combination for use according to any one of embodiments 1a to 15a, wherein the first query is associated with a first aspect of treatment of schizophrenia (e.g., auditory hallucinations) and the first set of digital therapies are associated with improving the first aspect of treatment of schizophrenia (e.g., improving voice control).

### Embodiments (b)

1b. A combination for use in the treatment of schizophrenia comprising an antipsychotic and a digital-therapeutic, wherein the digital-therapeutic delivers behavioral therapy.

2b. A combination for use in the treatment of positive symptoms of schizophrenia comprising an antipsychotic and a digital-therapeutic, wherein the digital-therapeutic delivers behavioral therapy.

3b. The combination for use according to embodiment 2b, wherein positive symptoms of schizophrenia are one or more selected from the group comprising delusions associated with schizophrenia, disorganized thinking associated with schizophrenia, hallucinations associated with schizophrenia, excitement associated with schizophrenia, grandiosity associated with schizophrenia, suspiciousness associated with schizophrenia and hostility associated with schizophrenia.

4b. A combination for use in the treatment of negative symptoms of schizophrenia comprising an antipsychotic and a digital-therapeutic, wherein the digital-therapeutic delivers behavioral therapy.

5b. The combination for use according to embodiment 4b, wherein negative symptoms of schizophrenia are one or selected from the group comprising blunted affect associated with schizophrenia, emotional withdrawal associated with schizophrenia, poor rapport associated with schizophrenia, apathetic social withdrawal associated with schizophrenia, difficulty in abstract thinking associated with schizophrenia, lack of spontaneity and flow of conversation associated with schizophrenia and stereotyped thinking associated with schizophrenia.

6b. A combination for use in the treatment of depression associated with schizophrenia comprising an antipsychotic and a digital-therapeutic, wherein the digital-therapeutic delivers behavioral therapy.

7b. A combination for use in the treatment of insomnia associated with schizophrenia comprising an antipsychotic and a digital-therapeutic, wherein the digital-therapeutic delivers behavioral therapy.

8b. The combination for use according to any one of embodiments 1b to 7b, wherein the digital-therapeutic is system-prompted or on-demand.

9b. The combination for use according to any one of embodiments 1b to 8b, wherein the digital-therapeutic comprises (alternatively, is implemented on) a mobile device selected from the group comprising a smartphone, a laptop computer, a tablet computer, and a wearable computer.

10b. The combination for use according to any one of embodiments lb to 9b, which comprises one or more active agent (i.e., at least one further active agent).

11 b. The combination for use according to embodiment 10b, wherein the antipsychotic and the one or more active agent (i.e., at least one further active agent) is in the form of a fixed pharmaceutical combination or a non-fixed pharmaceutical combination.

12b. The combination for use according to embodiments 10b or 11b, wherein the further active agent is selected from the group comprising an antidepressant and an anxiolytic.

13b. The combination for use according to any one of embodiments lb to 12b, wherein the behavioral therapy is cognitive behavioral therapy for schizophrenia.

14b. The combination for use according to embodiment 13b, wherein the cognitive behavioral therapy for schizophrenia focuses on symptoms of psychosis, mood management, sleep hygiene, medication adherence or social skills.

15b. The combination for use according to embodiment 13b, wherein the cognitive behavioral therapy for schizophrenia focuses on one or more of (i.e., on at least one of) i) to v): i) symptoms of psychosis, ii) mood management, iii) sleep hygiene, iv) medication adherence and v) social skills.

### Embodiments (c)

1c. An antipsychotic for use in the treatment of schizophrenia, wherein the use is combined with computer-implemented behavioral therapy, such as computer-implemented cognitive behavioral therapy for schizophrenia.

2c. An antipsychotic for use in the treatment of positive symptoms of schizophrenia, wherein the use is combined with computer-implemented behavioral therapy, such as computer-implemented cognitive behavioral therapy for schizophrenia.

3c. The antipsychotic for use according to embodiment 2c, wherein positive symptoms of schizophrenia are one or more selected from the group comprising delusions associated with schizophrenia, disorganized thinking associated with schizophrenia, hallucinations associated with schizophrenia, excitement associated with schizophrenia, grandiosity associated with schizophrenia, suspiciousness associated with schizophrenia and hostility associated with schizophrenia.

4c. An antipsychotic for use in the treatment of negative symptoms of schizophrenia, wherein the use is combined with computer-implemented behavioral therapy, such as computer-implemented cognitive behavioral therapy for schizophrenia.

5c. The antipsychotic for use according to embodiment 4c, wherein negative symptoms of schizophrenia are one or more selected from the group comprising blunted affect associated with schizophrenia, emotional withdrawal associated with schizophrenia, poor rapport associated with schizophrenia, apathetic social withdrawal associated with schizophrenia, difficulty in abstract thinking associated with schizophrenia, lack of spontaneity and flow of conversation associated with schizophrenia and stereotyped thinking associated with schizophrenia.

6c. An antipsychotic for use in the treatment of depression associated with schizophrenia, wherein the use is combined with computer-implemented behavioral therapy, such as computer-implemented cognitive behavioral therapy for schizophrenia.

7c. An antipsychotic for use in the treatment of insomnia associated with schizophrenia, wherein the use is combined with computer-implemented behavioral therapy, such as computer-implemented cognitive behavioral therapy for schizophrenia.

8c. The antipsychotic for use according to any one of embodiments 1c to 7c, wherein the computer-implemented behavioral therapy, such as computer-implemented cognitive behavioral therapy for schizophrenia, is system-prompted or on-demand.

9c. The antipsychotic for use according to any one of embodiments 1c to 8c, wherein the computer-implemented behavioral therapy, such as computer-implemented cognitive behavioral therapy for schizophrenia, focuses on symptoms of psychosis, mood management, sleep hygiene, medication adherence or social skills.

10c. The antipsychotic for use according to any one of embodiments 1c to 8c, wherein the computer-implemented behavioral therapy, such as computer-implemented cognitive behavioral therapy for schizophrenia, focuses on one or more of (i.e., on at least one of) i) to v): i) symptoms of psychosis, ii) mood management, iii) sleep hygiene, iv) medication adherence and v) social skills.

11c. The antipsychotic for use according to any one of embodiments 1c to 10c, wherein the computer-implemented behavioral therapy, such as computer-implemented cognitive behavioral therapy for schizophrenia, is provided by a computerized device.

12c. The antipsychotic for use according to embodiment 11c, wherein the computerized device is a mobile device.

13c. The antipsychotic for use according to embodiment 12c, wherein the mobile device is selected from the group comprising a smartphone, a laptop computer, a tablet computer, and a wearable computer.

14c. The antipsychotic for use according to any one of embodiments 1c to 13c, wherein the antipsychotic is in the form of a pharmaceutical combination comprising one or more active agent (i.e., at least one further active agent).

15c. The antipsychotic for use according to embodiment 14c, wherein the pharmaceutical combination is a fixed pharmaceutical combination or a non-fixed pharmaceutical combination.

16c. The antipsychotic for use according to embodiments 14c or 15c, wherein the one or more active agent (i.e., at least one further active agent) is selected from the group comprising an antidepressant and an anxiolytic.

### Embodiments (d)

1d. Use of an antipsychotic for the manufacture of a medicament for the treatment of schizophrenia, wherein
i) the medicament is provided in combination with a computer-implemented mobile device (e.g., the medicament is provided in combination with a code and/or computer-readable link for downloading a digital-therapeutic (e.g., an application)); and
ii) the computer-implemented mobile device comprises (e.g., comprises once the digital therapeutic is installed on a compatible electronic device (e.g., device 100)):
   a display;
   an input device;
   one or more processors; and
   memory storing one or more programs configured to be executed by the one or more processors, the one or more programs including instructions for:
      displaying, on the display, a query interface, the query interface presenting a plurality of queries, including a first query;
      while displaying the query interface, receiving, via the input device, a first sequence of inputs;
      in response to receiving the first sequence of inputs, displaying a response summary interface, the response summary interface including:
         in accordance with the response to the first query being a response of a first type, displaying, in a first region of the response summary interface, a first response element corresponding to the first query, the first response element including at least a first selectable graphical element corresponding to a first set of digital therapies associated with the first query; and
         in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

2d. Use of an antipsychotic for the manufacture of a medicament for treatment of positive symptoms of schizophrenia, wherein
i) the medicament is provided in combination with a computer-implemented mobile device; and
ii) the computer-implemented mobile device comprises:
   a display;
   an input device;

2one or more processors; and
memory storing one or more programs configured to be executed by the one or more processors, the one or more programs including instructions for:
   displaying, on the display, a query interface, the query interface presenting a plurality of queries, including a first query;
   while displaying the query interface, receiving, via the input device, a first sequence of inputs;
   in response to receiving the first sequence of inputs, displaying a response summary interface, the response summary interface including:
      in accordance with the response to the first query being a response of a first type, displaying, in a first region of the response summary interface, a first response element corresponding to the first query, the first response element including at least a first selectable graphical element corresponding to a first set of digital therapies associated with the first query; and
      in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

3d. The use according to embodiment 2d, wherein positive symptoms of schizophrenia are one or more selected from the group comprising delusions associated with schizophrenia, disorganized thinking associated with schizophrenia, hallucinations associated with schizophrenia, excitement associated with schizophrenia, grandiosity associated with schizophrenia, suspiciousness associated with schizophrenia and hostility associated with schizophrenia.

4d. Use of an antipsychotic for the manufacture of a medicament for treatment of negative symptoms of schizophrenia, wherein
i) the medicament is provided in combination with a computer-implemented mobile device; and
ii) the computer-implemented mobile device comprises:
   a display;
   an input device;
   one or more processors; and
   memory storing one or more programs configured to be executed by the one or more processors, the one or more programs including instructions for:
      displaying, on the display, a query interface, the query interface presenting a plurality of queries, including a first query;
      while displaying the query interface, receiving, via the input device, a first sequence of inputs;
      in response to receiving the first sequence of inputs, displaying a response summary interface, the response summary interface including:
         in accordance with the response to the first query being a response of a first type, displaying, in a first region of the response summary interface, a first response element corresponding to the first query, the first response element including at least a first selectable graphical element corresponding to a first set of digital therapies associated with the first query; and
         in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

5d. The use according to embodiment 4d, wherein negative symptoms of schizophrenia are one or selected from the group comprising blunted affect associated with schizophrenia, emotional withdrawal associated with schizophrenia, poor rapport associated with schizophrenia, apathetic social withdrawal associated with schizophrenia, difficulty in abstract thinking associated with schizophrenia, lack of spontaneity and flow of conversation associated with schizophrenia and stereotyped thinking associated with schizophrenia.

6d. Use of an antipsychotic for the manufacture of a medicament for treatment of depression associated with schizophrenia, wherein
i) the medicament is provided in combination with a computer-implemented mobile device; and
ii) the computer-implemented mobile device comprises:
   a display;
   an input device;
   one or more processors; and
   memory storing one or more programs configured to be executed by the one or more processors, the one or more programs including instructions for:
      displaying, on the display, a query interface, the query interface presenting a plurality of queries, including a first query;
      while displaying the query interface, receiving, via the input device, a first sequence of inputs;
      in response to receiving the first sequence of inputs, displaying a response summary interface, the response summary interface including:
         in accordance with the response to the first query being a response of a first type, displaying, in a first region of the response summary interface, a first response element corresponding to the first query, the first response element including at least a first selectable graphical element corresponding to a first set of digital therapies associated with the first query; and
         in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

7d. Use of an antipsychotic for the manufacture of a medicament for treatment of insomnia associated with schizophrenia, wherein
i) the medicament is provided in combination with a computer-implemented mobile device; and
ii) the computer-implemented mobile device comprises:
   a display;
   an input device;
   one or more processors; and
   memory storing one or more programs configured to be executed by the one or more processors, the one or more programs including instructions for:
      displaying, on the display, a query interface, the query interface presenting a plurality of queries, including a first query;
      while displaying the query interface, receiving, via the input device, a first sequence of inputs;
      in response to receiving the first sequence of inputs, displaying a response summary interface, the response summary interface including:
         in accordance with the response to the first query being a response of a first type, displaying, in a first region of the response summary interface, a first response element corresponding to the first query, the first response element including at least a first selectable graphical element corresponding to a first set of digital therapies associated with the first query; and
         in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

8d. The use according to any one of embodiments 1d to 7d, wherein the computer-implemented mobile device is system-prompted or on-demand.

9d. The use according to any one of embodiments 1d to 8d, wherein the computer-implemented mobile device is selected from the group comprising a smartphone, a laptop computer, a tablet computer and a wearable computer.

10d. The use according to any one of embodiments 1d to 9d, wherein the medicament comprises one or more active agent (i.e., at least one further active agent).

11d. The use according to embodiment 10d, wherein the medicament comprising one or more active agent (i.e., at least one further active agent) is a fixed pharmaceutical combination or a non-fixed pharmaceutical combination.

12d. The use according to embodiments 10d or 11d, wherein the further active agent is selected from the group comprising an antidepressant and an anxiolytic.

13d. The use according to any one of embodiments 1d to 12d, wherein the computer-implemented mobile device delivers behavioral therapy.

14d. The use according to embodiment 13d, wherein the behavioral therapy is cognitive behavioral therapy for schizophrenia, such as cognitive behavioral therapy for schizophrenia that focuses on symptoms of psychosis, mood management, sleep hygiene, medication adherence or social skills.

15d. The use according to embodiment 13d, wherein the behavioral therapy is cognitive behavioral therapy for schizophrenia, such as cognitive behavioral therapy for schizophrenia that focuses on one or more of (i.e., on at least one of) i) to v): i) symptoms of psychosis, ii) mood management, iii) sleep hygiene, iv) medication adherence and v) social skills.

16d. The use according to any one of embodiments 1d to 15d, wherein the first query is associated with a first symptom of schizophrenia and the first set of digital therapies is configured to address the first symptom of schizophrenia.

17d. The use according to any one of embodiments 1d to 15d, wherein the first query is associated with a first aspect of treatment of schizophrenia (e.g., auditory hallucinations) and the first set of digital therapies are associated with improving the first aspect of treatment of schizophrenia (e.g., improving voice control).

### Embodiments (e)

1e. Use of an antipsychotic for the manufacture of a medicament for the treatment of schizophrenia, wherein
i) the medicament is provided in combination with a digital-therapeutic; and
ii) the digital-therapeutic delivers behavioral therapy.

2e. Use of an antipsychotic for the manufacture of a medicament for treatment of positive symptoms of schizophrenia, wherein
i) the medicament is provided in combination with a digital-therapeutic; and
ii) the digital-therapeutic delivers behavioral therapy.

3e. The use according to embodiment 2e, wherein positive symptoms of schizophrenia are one or more selected from the group comprising delusions associated with schizophrenia, disorganized thinking associated with schizophrenia, hallucinations associated with schizophrenia, excitement associated with schizophrenia, grandiosity associated with schizophrenia, suspiciousness associated with schizophrenia and hostility associated with schizophrenia.

4e. Use of an antipsychotic for the manufacture of a medicament for treatment of negative symptoms of schizophrenia, wherein
i) the medicament is provided in combination with a digital-therapeutic; and
ii) the digital-therapeutic delivers behavioral therapy.

5e. The use according to embodiment 4e, wherein negative symptoms of schizophrenia are one or selected from the group comprising blunted affect associated with schizophrenia, emotional withdrawal associated with schizophrenia, poor rapport associated with schizophrenia, apathetic social withdrawal associated with schizophrenia, difficulty in abstract thinking associated with schizophrenia, lack of spontaneity and flow of conversation associated with schizophrenia and stereotyped thinking associated with schizophrenia.

6e. Use of an antipsychotic for the manufacture of a medicament for treatment of depression associated with schizophrenia, wherein

i) the medicament is provided in combination with a digital-therapeutic; and

ii) the digital-therapeutic delivers behavioral therapy.

7e. Use of an antipsychotic for the manufacture of a medicament for treatment of insomnia associated with schizophrenia, wherein

i) the medicament is provided in combination with a digital-therapeutic; and

ii) the digital-therapeutic delivers behavioral therapy.

8e. The use according to any one of embodiments le to 7e, wherein the digital-therapeutic is system-prompted or on-demand.

9e. The use according to any one of embodiments le to 8e, wherein the digital-therapeutic comprises (alternatively, is implemented on) a mobile device selected from the group comprising a smartphone, a laptop computer, a tablet computer, and a wearable computer.

10e. The use according to any one of embodiments 1 e to 9e, wherein the medicament comprises one or more active agent (i.e., at least one further active agent).

11e. The use according to embodiment 10e, wherein the medicament comprising one or more active agent (i.e., at least one further active agent) is a fixed pharmaceutical combination or a non-fixed pharmaceutical combination.

12e. The use according to embodiments 10e or 11e, wherein the further active agent is selected from the group comprising an antidepressant and an anxiolytic.

13e. The use according to any one of embodiments 1 e to 12e, wherein the behavioral therapy is cognitive behavioral therapy for schizophrenia.

14e. The use according to embodiment 13e, wherein the cognitive behavioral therapy for schizophrenia focuses on symptoms of psychosis, mood management, sleep hygiene, medication adherence or social skills.

15e. The use according to embodiment 13e, wherein the cognitive behavioral therapy for schizophrenia focuses on one or more of (i.e., on at least one of) i) to v): i) symptoms of psychosis, ii) mood management, iii) sleep hygiene, iv) medication adherence and v) social skills.

### Embodiments (f)

1f. Use of an antipsychotic for the manufacture of a medicament for the treatment of schizophrenia, wherein the use is combined with computer-implemented behavioral therapy, such as computer-implemented cognitive behavioral therapy for schizophrenia.

2f. Use of an antipsychotic for the manufacture of a medicament for treatment of positive symptoms of schizophrenia, wherein the use is combined with computer-implemented behavioral therapy, such as computer-implemented cognitive behavioral therapy for schizophrenia.

3f. The use according to embodiment 2f, wherein positive symptoms of schizophrenia are one or more selected from the group comprising delusions associated with schizophrenia, disorganized thinking associated with schizophrenia, hallucinations associated with schizophrenia, excitement associated with schizophrenia, grandiosity associated with schizophrenia, suspiciousness associated with schizophrenia and hostility associated with schizophrenia.

4f. Use of an antipsychotic for the manufacture of a medicament for the treatment of negative symptoms of schizophrenia, wherein the use is combined with computer-implemented behavioral therapy, such as computer-implemented cognitive behavioral therapy for schizophrenia.

5f. The use according to embodiment 4f, wherein negative symptoms of schizophrenia are one or more selected from the group comprising blunted affect associated with schizophrenia, emotional withdrawal associated with schizophrenia, poor rapport associated with schizophrenia, apathetic social withdrawal associated with schizophrenia, difficulty in abstract thinking associated with schizophrenia, lack of spontaneity and flow of conversation associated with schizophrenia and stereotyped thinking associated with schizophrenia.

6f. Use of an antipsychotic for the manufacture of a medicament for the treatment of depression associated with schizophrenia, wherein the use is combined with computer-implemented behavioral therapy, such as computer-implemented cognitive behavioral therapy for schizophrenia.

7f. Use of an antipsychotic for the manufacture of a medicament for the treatment of insomnia associated with schizophrenia, wherein the use is combined with computer-implemented behavioral therapy, such as computer-implemented cognitive behavioral therapy for schizophrenia.

8f. The use according to any one of embodiments 1f to 7f, wherein the computer-implemented behavioral therapy, such as computer-implemented cognitive behavioral therapy for schizophrenia, is system-prompted or on-demand.

9f. The use according to any one of embodiments 1f to 8f, wherein the computer-implemented behavioral therapy, such as computer-implemented cognitive behavioral therapy for schizophrenia, focuses on symptoms of psychosis, mood management, sleep hygiene, medication adherence or social skills.

10f. The use according to any one of embodiments 1f to 9f, wherein the computer-implemented behavioral therapy, such as computer-implemented cognitive behavioral therapy for schizophrenia, focuses on one or more of (i.e., on at least one of) i) to v): i) symptoms of psychosis, ii) mood management, iii) sleep hygiene, iv) medication adherence and v) social skills.

11f. The use according to any one of embodiments 1f to 10f, wherein the computer-implemented behavioral therapy, such as computer-implemented cognitive behavioral therapy for schizophrenia, is provided by a computerized device.

12f. The use according to embodiment 11f, wherein the computerized device is a mobile device.

13f. The use according to embodiment 12f, wherein the mobile device is selected from the group comprising a smartphone, a laptop computer, a tablet computer, and a wearable computer.

14f. The use according to any one of embodiments 1f to 13f, wherein the antipsychotic is in the form of a pharmaceutical combination comprising one or more active agent (i.e., at least one further active agent).

15f. The use according to embodiment 14f, wherein the pharmaceutical combination is a fixed pharmaceutical combination or a non-fixed pharmaceutical combination.

16f. The use according to embodiments 14f or 15f, wherein the one or more active agent (i.e., at least one further active agent) is selected from the group comprising an antidepressant and an anxiolytic.

### Embodiments (q)

1g. A method for the treatment of schizophrenia, in a subject, in need thereof, comprising administering to said subject an effective amount of an antipsychotic, wherein
i) the antipsychotic is provided in combination with a computer-implemented mobile device; and
ii) the computer-implemented mobile device comprises:
   a display;
   an input device;
   one or more processors; and
   memory storing one or more programs configured to be executed by the one or more processors, the one or more programs including instructions for:
      displaying, on the display, a query interface, the query interface presenting a plurality of queries, including a first query;
      while displaying the query interface, receiving, via the input device, a first sequence of inputs;
      in response to receiving the first sequence of inputs, displaying a response summary interface, the response summary interface including:
         in accordance with the response to the first query being a response of a first type, displaying, in a first region of the response summary interface, a first response element corresponding to the first query, the first response element including at least a first selectable graphical element corresponding to a first set of digital therapies associated with the first query; and
         in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

2g. A method for the treatment of positive symptoms of schizophrenia, in a subject, in need thereof, comprising administering to said subject an effective amount of an antipsychotic, wherein
i) the antipsychotic is provided in combination with a computer-implemented mobile device; and
ii) the computer-implemented mobile device comprises:
   a display;
   an input device;
   one or more processors; and
   memory storing one or more programs configured to be executed by the one or more processors, the one or more programs including instructions for:
      displaying, on the display, a query interface, the query interface presenting a plurality of queries, including a first query;
      while displaying the query interface, receiving, via the input device, a first sequence of inputs;
      in response to receiving the first sequence of inputs, displaying a response summary interface, the response summary interface including:
         in accordance with the response to the first query being a response of a first type, displaying, in a first region of the response summary interface, a first response element corresponding to the first query, the first response element including at least a first selectable graphical element corresponding to a first set of digital therapies associated with the first query; and
         in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

3g. The method according to embodiment 2g, wherein positive symptoms of schizophrenia are one or more selected from the group comprising delusions associated with schizophrenia, disorganized thinking associated with schizophrenia, hallucinations associated with schizophrenia, excitement associated with schizophrenia, grandiosity associated with schizophrenia, suspiciousness associated with schizophrenia and hostility associated with schizophrenia.

4g. A method for the treatment of negative symptoms of schizophrenia, in a subject, in need thereof, comprising administering to said subject an effective amount of an antipsychotic, wherein
i) the antipsychotic is provided in combination with a computer-implemented mobile device; and
ii) the computer-implemented mobile device comprises:
   a display;
   an input device;
   one or more processors; and
   memory storing one or more programs configured to be executed by the one or more processors, the one or more programs including instructions for:
      displaying, on the display, a query interface, the query interface presenting a plurality of queries, including a first query;
      while displaying the query interface, receiving, via the input device, a first sequence of inputs;
      in response to receiving the first sequence of inputs, displaying a response summary interface, the response summary interface including:
         in accordance with the response to the first query being a response of a first type, displaying, in a first region of the response summary interface, a first response element corresponding to the first query, the first response element including at least a first selectable graphical element corresponding to a first set of digital therapies associated with the first query; and
         in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

5g. The method according to embodiment 4g, wherein negative symptoms of schizophrenia are one or selected from the group comprising blunted affect associated with schizophrenia, emotional withdrawal associated with schizophrenia, poor rapport associated with schizophrenia, apathetic social withdrawal associated with schizophrenia, difficulty in abstract thinking associated with schizophrenia, lack of spontaneity and flow of conversation associated with schizophrenia and stereotyped thinking associated with schizophrenia.

6g. A method for treatment of depression associated with schizophrenia, in a subject, in need thereof, comprising administering to said subject an effective amount of an antipsychotic, wherein
i) the antipsychotic is provided in combination with a computer-implemented mobile device; and
ii) the computer-implemented mobile device comprises:
   a display;
   an input device;
   one or more processors; and
   memory storing one or more programs configured to be executed by the one or more processors, the one or more programs including instructions for:
      displaying, on the display, a query interface, the query interface presenting a plurality of queries, including a first query;
      while displaying the query interface, receiving, via the input device, a first sequence of inputs;
      in response to receiving the first sequence of inputs, displaying a response summary interface, the response summary interface including:
         in accordance with the response to the first query being a response of a first type, displaying, in a first region of the response summary interface, a first response element corresponding to the first query, the first response element including at least a first selectable graphical element corresponding to a first set of digital therapies associated with the first query; and
         in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

7g. A method for treatment of insomnia associated with schizophrenia, in a subject, in need thereof, comprising administering to said subject an effective amount of an antipsychotic, wherein
i) the antipsychotic is provided in combination with a computer-implemented mobile device; and
ii) the computer-implemented mobile device comprises:
   a display;
   an input device;
   one or more processors; and memory storing one or more programs configured to be executed by the one or more processors, the one or more programs including instructions for:
      displaying, on the display, a query interface, the query interface presenting a plurality of queries, including a first query;
      while displaying the query interface, receiving, via the input device, a first sequence of inputs;
      in response to receiving the first sequence of inputs, displaying a response summary interface, the response summary interface including:
         in accordance with the response to the first query being a response of a first type, displaying, in a first region of the response summary interface, a first response element corresponding to the first query, the first response element including at least a first selectable graphical element corresponding to a first set of digital therapies associated with the first query; and
         in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

8g. The method according to any one of embodiments 1g to 7g, wherein the computer-implemented mobile device is system-prompted or on-demand.

9g. The method according to any one of embodiments 1g to 8g, wherein the computer-implemented mobile device is selected from the group comprising a smartphone, a laptop computer, a tablet computer and a wearable computer.

10g. The method according to any one of embodiments 1g to 9g, wherein the antipsychotic is in the form of a pharmaceutical combination comprising one or more active agent (i.e., at least one further active agent).

11g. The method according to embodiment 10g, wherein the pharmaceutical combination is a fixed pharmaceutical combination or a non-fixed pharmaceutical combination.

12g. The method according to embodiments 10g or 11g, wherein the one or more active agent (i.e., at least one further active agent) is selected from the group comprising an antidepressant and an anxiolytic.

13g. The method according to any one of embodiments 1g to 12g, wherein the computer-implemented mobile device delivers behavioral therapy.

14g. The method according to embodiment 13g, wherein the behavioral therapy is cognitive behavioral therapy for schizophrenia, such as cognitive behavioral therapy for schizophrenia that focuses on symptoms of psychosis, mood management, sleep hygiene, medication adherence or social skills.

15g. The method according to embodiment 14g, wherein the behavioral therapy is cognitive behavioral therapy for schizophrenia, such as cognitive behavioral therapy for schizophrenia that focuses on one or more of (i.e., on at least one of) i) to v): i) symptoms of psychosis, ii) mood management, iii) sleep hygiene, iv) medication adherence and v) social skills.

16g. The method according to any one of the embodiments 1g to 15g, wherein the first query is associated with a first symptom of schizophrenia and the first set of digital therapies is configured to address the first symptom of schizophrenia.

17g. The method according to any one of the embodiments 1g to 15g, wherein the first query is associated with a first aspect of treatment of schizophrenia (e.g., auditory hallucinations) and the first set of digital therapies are associated with improving the first aspect of treatment of schizophrenia (e.g., improving voice control).

### Embodiments (h)

1h. A method for the treatment of schizophrenia, in a subject, in need thereof, comprising administering to said subject an effective amount of an antipsychotic, wherein
i) the antipsychotic is provided in combination with a digital-therapeutic; and
ii) the digital-therapeutic delivers behavioral therapy.

2h. A method for treatment of positive symptoms of schizophrenia, in a subject, in need thereof, comprising administering to said subject an effective amount of an antipsychotic, wherein
i) the antipsychotic is provided in combination with a digital-therapeutic; and
ii) the digital-therapeutic delivers behavioral therapy.

3h. The method according to embodiment 2h, wherein positive symptoms of schizophrenia are one or more selected from the group comprising delusions associated with schizophrenia, disorganized thinking associated with schizophrenia, hallucinations associated with schizophrenia, excitement associated with schizophrenia, grandiosity associated with schizophrenia, suspiciousness associated with schizophrenia and hostility associated with schizophrenia.

4h. A method for treatment of negative symptoms of schizophrenia, in a subject, in need thereof, comprising administering to said subject an effective amount of an antipsychotic, wherein
i) the antipsychotic is provided in combination with a digital-therapeutic; and
ii) the digital-therapeutic delivers behavioral therapy.

5h. The method according to embodiment 4h, wherein negative symptoms of schizophrenia are one or selected from the group comprising blunted affect associated with schizophrenia, emotional withdrawal associated with schizophrenia, poor rapport associated with schizophrenia, apathetic social withdrawal associated with schizophrenia, difficulty in abstract thinking associated with schizophrenia, lack of spontaneity and flow of conversation associated with schizophrenia and stereotyped thinking associated with schizophrenia.

6h. A method for treatment of depression associated with schizophrenia, in a subject, in need thereof, comprising administering to said subject an effective amount of an antipsychotic, wherein
i) the antipsychotic is provided in combination with a digital-therapeutic; and
ii) the digital-therapeutic delivers behavioral therapy.

7h. A method for treatment of insomnia associated with schizophrenia, in a subject, in need thereof, comprising administering to said subject an effective amount of an antipsychotic, wherein
i) the antipsychotic is provided in combination with a digital-therapeutic; and
ii) the digital-therapeutic delivers behavioral therapy.

8h. The method according to any one of embodiments 1 h to 7h, wherein the digital-therapeutic is system-prompted or on-demand.

9h. The method according to any one of embodiments 1 h to 8h, wherein the digital-therapeutic comprises (alternatively, is implemented on) a mobile device selected from the group comprising a smartphone, a laptop computer, a tablet computer, and a wearable computer.

10h. The method according to any one of embodiments 1h to 9h, wherein the antipsychotic is in the form of a pharmaceutical combination comprising one or more active agent (i.e., at least one further active agent).

11h. The method according to embodiment 10h, wherein the pharmaceutical combination is a fixed pharmaceutical combination or a non-fixed pharmaceutical combination.

12h. The method according to embodiments 10h or 11h, wherein the one or more active agent (i.e., at least one further active agent) is selected from the group comprising an antidepressant and an anxiolytic.

13h. The method according to any one of embodiments 1h to 12h, wherein the behavioral therapy is cognitive behavioral therapy for schizophrenia.

14h. The method according to embodiment 13h, wherein the cognitive behavioral therapy for schizophrenia focuses on symptoms of psychosis, mood management, sleep hygiene, medication adherence or social skills.

15h. The method according to embodiment 13h, wherein the cognitive behavioral therapy for schizophrenia focuses on one or more of (i.e., on at least one of) i) to v): i) symptoms of psychosis, ii) mood management, iii) sleep hygiene, iv) medication adherence and v) social skills.

### Embodiments (j)

1j. A method for the treatment of schizophrenia, in a subject, in need thereof, comprising administering to said subject an effective amount of an antipsychotic, wherein the method is combined with computer-implemented behavioral therapy, such as computer-implemented cognitive behavioral therapy for schizophrenia.

2j. A method for the treatment of positive symptoms of schizophrenia, in a subject, in need thereof, comprising administering to said subject an effective amount of an antipsychotic, wherein, the method is combined with computer-implemented behavioral therapy, such as computer-implemented cognitive behavioral therapy for schizophrenia.

3j. The method according to embodiment 2j, wherein positive symptoms of schizophrenia are one or more selected from the group comprising delusions associated with schizophrenia, disorganized thinking associated with schizophrenia, hallucinations associated with schizophrenia, excitement associated with schizophrenia, grandiosity associated with schizophrenia, suspiciousness associated with schizophrenia and hostility associated with schizophrenia.

4j. A method for the treatment of negative symptoms of schizophrenia, in a subject, in need thereof, comprising administering to said subject an effective amount of an antipsychotic, wherein, the method is combined with computer-implemented behavioral therapy, such as computer-implemented cognitive behavioral therapy for schizophrenia.

5j. The method according to embodiment 4j, wherein negative symptoms of schizophrenia are one or more selected from the group comprising blunted affect associated with schizophrenia, emotional withdrawal associated with schizophrenia, poor rapport associated with schizophrenia, apathetic social withdrawal associated with schizophrenia, difficulty in abstract thinking associated with schizophrenia, lack of spontaneity and flow of conversation associated with schizophrenia and stereotyped thinking associated with schizophrenia.

6j. A method for the treatment of depression associated with schizophrenia, in a subject, in need thereof, comprising administering to said subject an effective amount of an antipsychotic, wherein, the method is combined with computer-implemented behavioral therapy, such as computer-implemented cognitive behavioral therapy for schizophrenia.

7j. A method for the treatment of insomnia associated with schizophrenia, in a subject, in need thereof, comprising administering to said subject an effective amount of an antipsychotic, wherein, the method is combined with computer-implemented behavioral therapy, such as computer-implemented cognitive behavioral therapy for schizophrenia.

8j. The method according to any one of embodiments 1j to 7j, wherein the computer-implemented behavioral therapy, such as computer-implemented cognitive behavioral therapy for schizophrenia, is system-prompted or on-demand.

9j. The method according to any one of embodiments 1j to 8j, wherein the computer-implemented behavioral therapy, such as computer-implemented cognitive behavioral therapy for schizophrenia, focuses on symptoms of psychosis, mood management, sleep hygiene, medication adherence or social skills.

10j. The method according to any one of embodiments 1j to 8j, wherein the computer-implemented behavioral therapy, such as computer-implemented cognitive behavioral therapy for schizophrenia, focuses on one or more of (i.e., on at least one of) i) to v): i) symptoms of psychosis, ii) mood management, iii) sleep hygiene, iv) medication adherence and v) social skills.

11j. The method according to any one of embodiments 1j to 10j, wherein the computer-implemented behavioral therapy, such as computer-implemented cognitive behavioral therapy for schizophrenia, is provided by a computerized device.

12j. The method according to embodiment 11j, wherein the computerized device is a mobile device.

13j. The method according to embodiment 12j, wherein the mobile device is selected from the group comprising a smartphone, a laptop computer, a tablet computer, and a wearable computer.

14j. The method according to any one of embodiments 1j to 13j, wherein the antipsychotic is in the form of a pharmaceutical combination comprising one or more active agent (i.e., at least one further active agent).

15j. The method according to embodiment 14j, wherein the pharmaceutical combination is a fixed pharmaceutical combination or a non-fixed pharmaceutical combination.

16j. The method according to embodiments 14j or 15j, wherein the one or more active agent (i.e., at least one further active agent) is selected from the group comprising an antidepressant and an anxiolytic.

### Embodiments (k)

1k. A computerized method for targeted delivery of digital therapies, the method comprising:

at an electronic device including a display and an input device:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
   in accordance with the response to the first query being a response of a first type, displaying, in a first region of the response summary interface, a first response element corresponding to the first query, the first response element including at least a first selectable graphical element corresponding to a first set of digital therapies associated with the first query; and
   in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

2k. The computerized method of embodiment 1k, wherein the plurality of queries includes a second query and the first sequence of inputs includes a response to the second query, the method further comprising:
further in response to the first sequence of inputs:
   in accordance with the response to the second query being a response of the first type, displaying, in the first region of the response summary interface, a third response element corresponding to the second query, the third response element including at least a second selectable graphical element corresponding to a second set of digital therapies associated with the second query; and
   in accordance with the response to the second query being a response of the second type, displaying, in the second region of the response summary interface, a fourth response element, wherein the fourth response element corresponds to the second query and does not include the second selectable graphical element corresponding to the second set of digital therapies associated with the second query.

3k. The computerized method of any of embodiments 1 k-2k, the method further comprising:
while displaying the first response element, receiving a second sequence of inputs, including an input corresponding to selection of the first selectable element;
in response to receiving the second sequence of inputs, displaying a first set of selectable graphical elements corresponding to the first set of digital therapies, including at least a third selectable graphical element corresponding to a first digital therapy associated with the first query;
while displaying the first set of digital therapies, receiving a third sequence of inputs, including an input corresponding to selection of the third selectable graphical element corresponding to the first digital therapy; and
in response to receiving the third sequence of inputs, outputting the first digital therapy, the first digital therapy including at least a multimedia component.

4k. The computerized method of embodiment 3k, the method further comprising:
after outputting the first digital therapy, displaying an option to categorize the first digital therapy as a designated digital therapy;
receiving an input corresponding to a selection of the option to categorize the first digital therapy as a designated digital therapy; and
in response to receiving the input corresponding to the selection of the option to categorize the first digital therapy as a designated digital therapy, categorizing the first digital therapy as a designated digital therapy.

5k. The computerized method of embodiment 4k, the method further comprising:
after categorizing the first digital therapy as a designated digital therapy, receiving a request to display a designated digital therapy interface;
in response to the request to display the designated digital therapy interface, displaying the designated digital therapy interface, the designated digital therapy interface including at least a fourth selectable graphical element corresponding to the designated first digital therapy.

6k. The computerized method of any of embodiments 1 k-5k, the method further comprising:
prior to displaying the query interface, displaying a home interface, the home interface including a second set of selectable graphical elements, the second set of selectable graphical elements including at least a selectable graphical element configured to cause display of the query interface; and
while displaying the home interface, receiving an input corresponding to selection of the selectable graphical element configured to cause display of the query interface,
wherein displaying the query interface occurs in response to receiving the input corresponding to selection of the selectable graphical element configured to cause display of the query interface.

7k. The computerized method of embodiment 6k, wherein the home interface further includes a selectable graphical element configured to cause display of a digital therapies interface, the method further including:
receiving an input corresponding to selection of the selectable graphical element configured to cause display of the digital therapies interface;
in response to receiving the input corresponding to selection of the selectable graphical element configured to cause display of the digital therapies interface, displaying the digital therapies interface, wherein the digital therapies interface includes at least:
   a fifth selectable graphical element corresponding to the first set of digital therapies; and
   a sixth selectable graphical element corresponding to the second set of digital therapies.

8k. The computerized methods of any of embodiments 1 k-7k, wherein the first query is associated with a first symptom of schizophrenia and the first set of digital therapies is configured to address the first symptom of schizophrenia.

9k. The computerized method of any of embodiments 1 k-8k, the method further comprising:
determining whether a plurality of sets of criteria are met;
in accordance with a determination that a first set of criteria the plurality of sets of criteria are met, outputting a notification of a first type; and
in accordance with a determination that a second set of criteria of the plurality of sets of criteria are met, the second set of criteria being different than the first set of criteria, outputting a notification of a second type, different than the first type.

10k. The computerized method of embodiment 9k, the method further comprising:
while the notification of the first type is displayed, receiving an input corresponding to selection of the notification of the first type;
in response to receiving the input corresponding to selection of the notification of the first type; displaying a third query and a third set of selectable graphical elements corresponding to a plurality of responses to the third query;
receiving a fourth sequence of inputs, including an input corresponding to selection of a selectable graphical element of the third set of selectable graphical elements;
in response to receiving the fourth sequence of inputs:
   in accordance with the fourth sequence of inputs including a selection of a first selectable graphical element of the third set of selectable graphical elements, displaying a first response interface that includes a selectable graphical element configured to cause display of the query interface and a selectable graphical element configured to cause display of the digital therapies interface; and
   in accordance with the fourth sequence of inputs including a selection of a second selectable graphical element of the third set of selectable graphical elements, displaying a second response interface that includes a selectable graphical element configured to cause display of the digital therapies interface.

11k. The computerized method of any of embodiments 1k-10k, the method further comprising:
while the notification of the second type is displayed, receiving an input corresponding to selection of the notification of the second type;
in response to receiving the input corresponding to selection of the notification of the second type, displaying a first interstitial interface;
after displaying the first interstitial interface for a predetermined period of time, transitioning the first interstitial interface to a second interstitial interface, wherein the second interstitial interface includes a selectable graphical element for causing display of a fourth query;
while displaying the second interstitial interface, receiving an input corresponding to selection of the selectable graphical element for causing display of the fourth query; and in response to receiving the input corresponding to selection of the selectable graphical element for causing display of a fourth query, displaying the fourth query.

12k. The computerized method according to any one of the embodiments 1k to 11k, wherein the first query is associated with a first symptom of schizophrenia and the first set of digital therapies is configured to address the first symptom of schizophrenia.

13K. The computerized method according to any one of the embodiments 1k to 11k, wherein the first query is associated with a first aspect of treatment of schizophrenia (e.g., auditory hallucinations) and the first set of digital therapies are associated with improving the first aspect of treatment of schizophrenia (e.g., improving voice control).

14k. A non-transitory computer-readable storage medium storing one or more programs configured to be executed by one or more processors of an electronic device with a display and a input device, the one or more programs including instructions for performing the method of any of embodiments 1k-13k.

15k. An electronic device, comprising:
a display;
an input device;
one or more processors; and
memory storing one or more programs configured to be executed by the one or more processors, the one or more programs including instructions for performing the method of any of embodiments 1k-13k.

16k. An electronic device, comprising:
a display;
an input device; and
means for performing the method of any of embodiments 1k-13k.

17k. A non-transitory computer-readable storage medium storing one or more programs configured to be executed by one or more processors of an electronic device with a display and an input device, the one or more programs including instructions for:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
   in accordance with the response to the first query being a response of a first type, displaying, in a first region of the response summary interface, a first response element corresponding to the first query, the first response element including at least a first selectable graphical element corresponding to a first set of digital therapies associated with the first query; and
   in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

18k. An electronic device, comprising:
a display;
an input device;
one or more processors; and
memory storing one or more programs configured to be executed by the one or more processors, the one or more programs including instructions for:
   displaying, on the display, a query interface, the query interface presenting a plurality of queries, including a first query;
   while displaying the query interface, receiving, via the input device, a first sequence of inputs;
   in response to receiving the first sequence of inputs, displaying a response summary interface, the response summary interface including:
      in accordance with the response to the first query being a response of a first type, displaying, in a first region of the response summary interface, a first response element corresponding to the first query, the first response element including at least a first selectable graphical element corresponding to a first set of digital therapies associated with the first query; and
      in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

### Further Embodiments

1. A method for the treatment of schizophrenia, in a subject, in need thereof, comprising administering to said subject an effective amount of an antipsychotic, wherein
i) the antipsychotic is provided in combination with a computer-implemented mobile device; and
ii) the computer-implemented mobile device comprises:
a display;
an input device;
one or more processors; and
memory storing one or more programs configured to be executed by the one or more processors, the one or more programs including instructions for:
   displaying, on the display, a query interface, the query interface presenting a plurality of queries, including a first query;
   while displaying the query interface, receiving, via the input device, a first sequence of inputs;
   in response to receiving the first sequence of inputs, displaying a response summary interface, the response summary interface including:
      in accordance with the response to the first query being a response of a first type, displaying, in a first region of the response summary interface, a first response element corresponding to the first query, the first response element including at least a first selectable graphical element corresponding to a first set of digital therapies associated with the first query; and
      in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

2. A method for the treatment of positive symptoms of schizophrenia, in a subject, in need thereof, comprising administering to said subject an effective amount of an antipsychotic, wherein,
i) the antipsychotic is provided in combination with a computer-implemented mobile device; and
ii) the computer-implemented mobile device comprises:
   a display;
   an input device;
   one or more processors; and
   memory storing one or more programs configured to be executed by the one or more processors, the one or more programs including instructions for:
      displaying, on the display, a query interface, the query interface presenting a plurality of queries, including a first query;
      while displaying the query interface, receiving, via the input device, a first sequence of inputs;
      in response to receiving the first sequence of inputs, displaying a response summary interface, the response summary interface including:
         in accordance with the response to the first query being a response of a first type, displaying, in a first region of the response summary interface, a first response element corresponding to the first query, the first response element including at least a first selectable graphical element corresponding to a first set of digital therapies associated with the first query; and
         in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

3. A method for the treatment of negative symptoms of schizophrenia, in a subject, in need thereof, comprising administering to said subject an effective amount of an antipsychotic, wherein,
i) the antipsychotic is provided in combination with a computer-implemented mobile device; and
ii) the computer-implemented mobile device comprises:
a display;
an input device;
one or more processors; and
memory storing one or more programs configured to be executed by the one or more processors, the one or more programs including instructions for:
   displaying, on the display, a query interface, the query interface presenting a plurality of queries, including a first query;
   while displaying the query interface, receiving, via the input device, a first sequence of inputs;
   in response to receiving the first sequence of inputs, displaying a response summary interface, the response summary interface including:
      in accordance with the response to the first query being a response of a first type, displaying, in a first region of the response summary interface, a first response element corresponding to the first query, the first response element including at least a first selectable graphical element corresponding to a first set of digital therapies associated with the first query; and
      in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

4. A method for treatment of depression associated with schizophrenia, in a subject, in need thereof, comprising administering to said subject an effective amount of an antipsychotic, wherein,
i) the antipsychotic is provided in combination with a computer-implemented mobile device; and
ii) the computer-implemented mobile device comprises:
a display;
an input device;
one or more processors; and
memory storing one or more programs configured to be executed by the one or more processors, the one or more programs including instructions for:
   displaying, on the display, a query interface, the query interface presenting a plurality of queries, including a first query;
   while displaying the query interface, receiving, via the input device, a first sequence of inputs;
   in response to receiving the first sequence of inputs, displaying a response summary interface, the response summary interface including:
      in accordance with the response to the first query being a response of a first type, displaying, in a first region of the response summary interface, a first response element corresponding to the first query, the first response element including at least a first selectable graphical element corresponding to a first set of digital therapies associated with the first query; and
      in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

5. A method for treatment of insomnia associated with schizophrenia, in a subject, in need thereof, comprising administering to said subject an effective amount of an antipsychotic, wherein,
i) the antipsychotic is provided in combination with a computer-implemented mobile device; and
ii) the computer-implemented mobile device comprises:
a display;
an input device;
one or more processors; and
memory storing one or more programs configured to be executed by the one or more processors, the one or more programs including instructions for:
   displaying, on the display, a query interface, the query interface presenting a plurality of queries, including a first query;
   while displaying the query interface, receiving, via the input device, a first sequence of inputs;
   in response to receiving the first sequence of inputs, displaying a response summary interface, the response summary interface including:
      in accordance with the response to the first query being a response of a first type, displaying, in a first region of the response summary interface, a first response element corresponding to the first query, the first response element including at least a first selectable graphical element corresponding to a first set of digital therapies associated with the first query; and
      in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

6. The method according to any one of embodiments 1 to 5, wherein the computer-implemented mobile device delivers behavioral therapy, such as cognitive behavioral therapy, for example cognitive behavioral therapy for schizophrenia that focuses on symptoms of psychosis, mood management, sleep hygiene, medication adherence or social skills.

7. The method according to embodiment 6, wherein the behavioral therapy is cognitive behavioral therapy for schizophrenia, such as cognitive behavioral therapy for schizophrenia that focuses on one or more of (i.e., on at least one of) i) to v): i) symptoms of psychosis, ii) mood management, iii) sleep hygiene, iv) medication adherence and v) social skills.

8. A method for the treatment of schizophrenia, in a subject, in need thereof, comprising administering to said subject an effective amount of an antipsychotic, wherein
i) the antipsychotic is provided in combination with a digital-therapeutic; and
ii) the digital-therapeutic delivers behavioral therapy.

9. A method for treatment of positive symptoms of schizophrenia, in a subject, in need thereof, comprising administering to said subject an effective amount of an antipsychotic, wherein
i) the antipsychotic is provided in combination with a digital-therapeutic; and
ii) the digital-therapeutic delivers behavioral therapy.

10. A method for treatment of negative symptoms of schizophrenia, in a subject, in need thereof, comprising administering to said subject an effective amount of an antipsychotic, wherein
i) the antipsychotic is provided in combination with a digital-therapeutic; and
ii) the digital-therapeutic delivers behavioral therapy.

11. A method for treatment of depression associated with schizophrenia, in a subject, in need thereof, comprising administering to said subject an effective amount of an antipsychotic, wherein
i) the antipsychotic is provided in combination with a digital-therapeutic; and
ii) the digital-therapeutic delivers behavioral therapy.

12. A method for treatment of insomnia associated with schizophrenia, in a subject, in need thereof, comprising administering to said subject an effective amount of an antipsychotic, wherein
i) the antipsychotic is provided in combination with a digital-therapeutic; and
ii) the digital-therapeutic delivers behavioral therapy.

13. The method according to any one of embodiments 8 to 12, wherein the digital-therapeutic comprises a mobile device selected from the group comprising a smartphone, a laptop computer, a tablet computer, and a wearable computer.

14. A method for the treatment of schizophrenia, in a subject, in need thereof, comprising administering to said subject an effective amount of an antipsychotic, wherein the method is combined with computer-implemented behavioral therapy, such as computer-implemented cognitive behavioral therapy for schizophrenia.

15. A method for the treatment of positive symptoms of schizophrenia, in a subject, in need thereof, comprising administering to said subject an effective amount of an antipsychotic, wherein, the method is combined with computer-implemented behavioral therapy, such as computer-implemented cognitive behavioral therapy for schizophrenia.

16. A method for the treatment of negative symptoms of schizophrenia, in a subject, in need thereof, comprising administering to said subject an effective amount of an antipsychotic, wherein, the method is combined with computer-implemented behavioral therapy, such as computer-implemented cognitive behavioral therapy for schizophrenia.

17. A method for the treatment of depression associated with schizophrenia, in a subject, in need thereof, comprising administering to said subject an effective amount of an antipsychotic, wherein, the method is combined with computer-implemented behavioral therapy, such as computer-implemented cognitive behavioral therapy for schizophrenia.

18. A method for the treatment of insomnia associated with schizophrenia, in a subject, in need thereof, comprising administering to said subject an effective amount of an antipsychotic, wherein, the method is combined with computer-implemented behavioral therapy, such as computer-implemented cognitive behavioral therapy for schizophrenia.

19. The method according to any one of embodiments 14 to 18, wherein the computer-implemented behavioral therapy, such as computer-implemented cognitive behavioral therapy for schizophrenia, is provided by a computerized device, such as a mobile device, for example, selected from the group comprising a smartphone, a laptop computer, a tablet computer, and a wearable computer.

20. The method according to any one of embodiments 14 to 19, wherein the antipsychotic is in the form of a pharmaceutical combination comprising one or more active agent (i.e., at least one further active agent).

21. The method according to embodiment 20, wherein the pharmaceutical combination is a fixed pharmaceutical combination or a non-fixed pharmaceutical combination.

22. The method according to embodiments 20 or 21, wherein the one or more active agent (i.e., at least one further active agent) is selected from the group comprising an antidepressant and an anxiolytic.

23. A combination comprising an antipsychotic and a computer-implemented mobile device; wherein the computer-implemented mobile device comprises:
a display;
an input device;
one or more processors; and
memory storing one or more programs configured to be executed by the one or more processors, the one or more programs including instructions for:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
   in accordance with the response to the first query being a response of a first type, displaying, in a first region of the response summary interface, a first response element corresponding to the first query, the first response element including at least a first selectable graphical element corresponding to a first set of digital therapies associated with the first query; and
   in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

24. A combination comprising an antipsychotic and a digital-therapeutic, wherein the digital-therapeutic delivers behavioral therapy, such as cognitive behavioral therapy for schizophrenia.

25. A computerized method for targeted delivery of digital therapies, the method comprising:

at an electronic device including a display and an input device:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
   in accordance with the response to the first query being a response of a first type, displaying, in a first region of the response summary interface, a first response element corresponding to the first query, the first response element including at least a first selectable graphical element corresponding to a first set of digital therapies associated with the first query; and
   in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

26. The computerized method of embodiment 25, wherein the plurality of queries includes a second query and the first sequence of inputs includes a response to the second query, the method further comprising:
further in response to the first sequence of inputs:
   in accordance with the response to the second query being a response of the first type, displaying, in the first region of the response summary interface, a third response element corresponding to the second query, the third response element including at least a second selectable graphical element corresponding to a second set of digital therapies associated with the second query; and
   in accordance with the response to the second query being a response of the second type, displaying, in the second region of the response summary interface, a fourth response element, wherein the fourth response element corresponds to the second query and does not include the second selectable graphical element corresponding to the second set of digital therapies associated with the second query.

27. The computerized method of any of embodiments 25-26, the method further comprising:
while displaying the first response element, receiving a second sequence of inputs, including an input corresponding to selection of the first selectable element;
in response to receiving the second sequence of inputs, displaying a first set of selectable graphical elements corresponding to the first set of digital therapies, including at least a third selectable graphical element corresponding to a first digital therapy associated with the first query;
while displaying the first set of digital therapies, receiving a third sequence of inputs, including an input corresponding to selection of the third selectable graphical element corresponding to the first digital therapy; and
in response to receiving the third sequence of inputs, outputting the first digital therapy, the first digital therapy including at least a multimedia component.

28. The computerized method of embodiment 27, the method further comprising:
after outputting the first digital therapy, displaying an option to categorize the first digital therapy as a designated digital therapy;
receiving an input corresponding to a selection of the option to categorize the first digital therapy as a designated digital therapy; and
in response to receiving the input corresponding to the selection of the option to categorize the first digital therapy as a designated digital therapy, categorizing the first digital therapy as a designated digital therapy.

29. The computerized method of embodiment 28, the method further comprising:
after categorizing the first digital therapy as a designated digital therapy, receiving a request to display a designated digital therapy interface;
in response to the request to display the designated digital therapy interface, displaying the designated digital therapy interface, the designated digital therapy interface including at least a fourth selectable graphical element corresponding to the designated first digital therapy.

30. The computerized method of any of embodiments 25-29, the method further comprising:
prior to displaying the query interface, displaying a home interface, the home interface including a second set of selectable graphical elements, the second set of selectable graphical elements including at least a selectable graphical element configured to cause display of the query interface; and
while displaying the home interface, receiving an input corresponding to selection of the selectable graphical element configured to cause display of the query interface,
wherein displaying the query interface occurs in response to receiving the input corresponding to selection of the selectable graphical element configured to cause display of the query interface.

31. The computerized method of embodiment 30, wherein the home interface further includes a selectable graphical element configured to cause display of a digital therapies interface, the method further including:
receiving an input corresponding to selection of the selectable graphical element configured to cause display of the digital therapies interface;
in response to receiving the input corresponding to selection of the selectable graphical element configured to cause display of the digital therapies interface, displaying the digital therapies interface, wherein the digital therapies interface includes at least:
   a fifth selectable graphical element corresponding to the first set of digital therapies; and
   a sixth selectable graphical element corresponding to the second set of digital therapies.

32. The computerized methods of any of embodiments 25-31, wherein the first query is associated with a first symptom of schizophrenia and the first set of digital therapies is configured to address the first symptom of schizophrenia.

33. The computerized method of any of embodiments 25-32, the method further comprising:
determining whether a plurality of sets of criteria are met;
in accordance with a determination that a first set of criteria the plurality of sets of criteria are met, outputting a notification of a first type; and
in accordance with a determination that a second set of criteria of the plurality of sets of criteria are met, the second set of criteria being different than the first set of criteria, outputting a notification of a second type, different than the first type.

34. The computerized method of embodiment 33, the method further comprising:
while the notification of the first type is displayed, receiving an input corresponding to selection of the notification of the first type;
in response to receiving the input corresponding to selection of the notification of the first type; displaying a third query and a third set of selectable graphical elements corresponding to a plurality of responses to the third query;
receiving a fourth sequence of inputs, including an input corresponding to selection of a selectable graphical element of the third set of selectable graphical elements;
in response to receiving the fourth sequence of inputs:
   in accordance with the fourth sequence of inputs including a selection of a first selectable graphical element of the third set of selectable graphical elements, displaying a first response interface that includes a selectable graphical element configured to cause display of the query interface and a selectable graphical element configured to cause display of the digital therapies interface; and
   in accordance with the fourth sequence of inputs including a selection of a second selectable graphical element of the third set of selectable graphical elements, displaying a second response interface that includes a selectable graphical element configured to cause display of the digital therapies interface.

35. The computerized method of any of embodiments 25-34, the method further comprising:
while the notification of the second type is displayed, receiving an input corresponding to selection of the notification of the second type;
in response to receiving the input corresponding to selection of the notification of the second type, displaying a first interstitial interface;
after displaying the first interstitial interface for a predetermined period of time, transitioning the first interstitial interface to a second interstitial interface, wherein the second interstitial interface includes a selectable graphical element for causing display of a fourth query;
while displaying the second interstitial interface, receiving an input corresponding to selection of the selectable graphical element for causing display of the fourth query; and
in response to receiving the input corresponding to selection of the selectable graphical element for causing display of a fourth query, displaying the fourth query.

36. A non-transitory computer-readable storage medium storing one or more programs configured to be executed by one or more processors of an electronic device with a display and a input device, the one or more programs including instructions for performing the method of any of embodiments 25-35.

37. An electronic device, comprising:
a display;
an input device;
one or more processors; and
memory storing one or more programs configured to be executed by the one or more processors, the one or more programs including instructions for performing the method of any of embodiments 25-35.

38. An electronic device, comprising:
a display;
an input device; and
means for performing the method of any of embodiments 25-35.

39. A non-transitory computer-readable storage medium storing one or more programs configured to be executed by one or more processors of an electronic device with a display and an input device, the one or more programs including instructions for:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
   in accordance with the response to the first query being a response of a first type, displaying, in a first region of the response summary interface, a first response element corresponding to the first query, the first response element including at least a first selectable graphical element corresponding to a first set of digital therapies associated with the first query; and
   in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

40. An electronic device, comprising:
a display;
an input device;
one or more processors; and
memory storing one or more programs configured to be executed by the one or more processors, the one or more programs including instructions for:
   displaying, on the display, a query interface, the query interface presenting a plurality of queries, including a first query;
   while displaying the query interface, receiving, via the input device, a first sequence of inputs;
   in response to receiving the first sequence of inputs, displaying a response summary interface, the response summary interface including:
      in accordance with the response to the first query being a response of a first type, displaying, in a first region of the response summary interface, a first response element corresponding to the first query, the first response element including at least a first selectable graphical element corresponding to a first set of digital therapies associated with the first query; and
      in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

General Terms

The term “schizophrenia” or “SZ”, as used herein, is defined, for example, with reference to DSM-5 criteria (i.e., according to the Diagnostic and Statistical Manual of Mental Disorders, 5^{th} Edition, Washington, DC: American Psychiatric Association, 2013), the entire contents of which are incorporated herein by reference. The DSM-5 outlines the following criterion to make a diagnosis of schizophrenia [i.e., DMS-5 diagnostic criteria 295.90 (F20.90)]:

A. Two (or more) of the following, each present for a significant portion of time during a 1-month period. And, at least one of these must be (A1), (A2), or (A3):
A1 Delusions.
A2 Hallucinations.
A3 Disorganized speech (e.g., frequent derailment or incoherence).
A4 Grossly disorganized or catatonic behavior.
A5 Negative symptoms [e.g., e.g., diminished emotional expression or avolition (i.e., reduced drive to pursue goal-directed behavior)].

B. For a significant portion of the time since the onset of the disturbance, level of functioning in one or more major areas (e.g., work, interpersonal relations, or self-care) is markedly below the level achieved prior to the onset (or when the onset is in childhood or adolescence, there is failure to achieve expected level of interpersonal, academic, or occupational functioning).

C. Continuous signs of the disturbance persist for at least 6 months. This 6-month period must include at least 1 month of symptoms that meet Criterion A (i.e., active-phase symptoms) and may include periods of prodromal or residual symptoms. During these prodromal or residual periods, the signs of the disturbance may be manifested by only negative symptoms or by two or more symptoms listed in Criterion A, present in an attenuated form (e.g., odd beliefs, unusual perceptual experiences).

D. Schizoaffective disorder and depressive or bipolar disorder with psychotic features have been ruled out because either 1) no major depressive or manic episodes have occurred concurrently with the active-phase symptoms, or 2) if mood episodes have occurred during active-phase symptoms, they have been present for a minority of the total duration of the active and residual periods of the illness.

E. The disturbance is not attributable to the physiological effects of a substance (e.g., a drug of abuse, a medication) or another medical condition.

F. If there is a history of autism spectrum disorder or a communication disorder of childhood onset, the additional diagnosis of schizophrenia is made only if prominent delusions or hallucinations, in addition to the other required symptoms of schizophrenia, are also present for at least 1 month.

The term “for at least”, for example in the expression “for at least one month”, means for one month or more.

The term “schizophrenic patient” or “patient with schizophrenia” refers to a patient diagnosed with schizophrenia, as defined herein. I

The term “PANSS” refers to the Positive and Negative Syndrome Scale of Schizophrenia (i.e., as defined in Kay S. R., Fizbein A., Opler L. A., Schizophrenia Bulletin, 1987, 13: 261-76; or in Depp, C. A., Loughran, C., Vahia, I., Molinari, V., Handbook of Assessment in Clinical Gerontology, 2010, Elsveier Inc., Chapter 5). It is a standardized 30 items clinical interview that rates the presence and severity of positive symptoms (7 items, i.e., namely items P1 to P7, of the Positive Scale (P) as defined herein below), negative symptoms (7 items, i.e., namely items N1 to N7, of the Negative Scale (N) as defined herein below), as well as general psychopathology (16 items, i.e., namely items G1 to G16, of the General Psychopathology Scale (G) as defined herein below) of patients with schizophrenia. The General Psychopathology Scale (G) is an adjunct to the positive and negative assessments, since it provides a separate but parallel measure of severity of schizophrenia that can serve as point reference, or control measure, for interpreting the syndromal scores.

For each of the 30 items, detailed anchoring criteria for 1 to 7 rating points is provided. The rating points represent increasing levels of psychophatology, as follows:

1=absent;

2=minimal (i.e., questionable or subtle or suspected pathology, or extreme end of the normal range);

3=mild (i.e., indicative of a symptom whose presence is clearly established but not pronounced and interferes little in day-today functioning);

4=moderate (i.e., symptom which, though representing a serious problem, either occurs only occasionally or intrudes on daily life only to a moderate extent);

5=moderate severe (i.e., marked manifestations that distinctly impact on one's functioning but are not all-consuming and usually can be contained at will);

6=severe (i.e., gross pathology that is present very frequently, proves highly disruptive to one's life, and often calls for direct supervision); and

7=extreme (i.e., the most serious level of psychopathology, whereby the manifestations drastically interfere in most or all major life functions, typically necessitating close supervision and assistance in many areas).

The scores for these scales are arrived at by summation of ratings across component items.

Positive Scale (P)

P1. Delusions: Beliefs which are unfounded, unrealistic, and idiosyncratic.

Basis for rating (i.e., rating points 1-7 as herein): Thought content expressed in the interview and its influence on social relations and behavior.

1 Absent—Definition does not apply.

2 Minimal—Questionable pathology; may be at the upper extreme of normal limits.

3 Mild—Presence of one or two delusions which are vague, uncrystallized, and not tenaciously held. Delusions do not interfere with thinking, social relations, or behavior.

4 Moderate—Presence of either a kaleidoscopic array of poorly-formed, unstable delusions or of a few well-formed delusions that occasionally interfere with thinking, social relations, or behavior.

5 Moderate severe—Presence of numerous well-formed delusions that are tenaciously held and occasionally interfere with thinking, social relations, or behavior.

6 Severe—Presence of a stable set of delusions which are crystallized, possibly systematized, tenaciously held, and clearly interfere with thinking, social relations, and behavior.

7 Extreme—Presence of a stable set of delusions which are either highly systematized or very numerous, and which dominate major facets of the patient's life. This frequently results in inappropriate and irresponsible action, which may even jeopardize the safety of the patient or others.

P2. Conceptual disorganization: Disorganized process of thinking characterized by disruption of goal-directed sequencing (e.g., circumstantiality, tangentiality, gross illogicality, or thought block).

Basis for rating (i.e., rating points 1-7 as herein): Cognitive-verbal processes observed during the course of interview.

1 Absent—Definition does not apply. 2 Minimal—Questionable pathology; may be at the upper extreme of normal limits.

3 Mild—Thinking is circumstantial, tangential, or paralogical. There is some difficulty in directing thoughts toward a goal and some loosening of associations may be evidenced under pressure.

4 Moderate—Able to focus thoughts when communications are brief and structured, but becomes loose or irrelevant when dealing with more complex communications or when under minimal pressure. 5 Moderate severe—Generally has difficulty in organizing thoughts, as evidenced by frequent irrelevances, disconnectedness. or loosening of associations even when not under pressure. 6 Severe—Thinking is seriously derailed and internally inconsistent, resulting in gross irrelevancies and disruption of thought processes, which occur almost constantly.

7 Extreme—Thoughts are disrupted to the point where the patient is incoherent. There is marked loosening of associations, which results in total failure of communication, e.g., “word salad” or mutism.

P3. Hallucinatory behavior: Verbal report or behavior indicating perceptions which are not generated by external stimuli. These may occur in the auditory, visual, olfactory, or somatic realms (i.e., auditory hallucinations, visual hallucinations, olfactory hallucinations or somatic hallucinations, respectively). As used herein, the term “hallucinatory behavior” refers to hallucinations, for example, auditory hallucinations, visual hallucinations, olfactory hallucinations or somatic hallucinations, such as visual hallucinations, olfactory hallucinations or somatic hallucinations.

Basis for rating (i.e., rating points 1-7 as herein): Verbal report and physical manifestations during the course of interview, as well as reports of behavior by primary care workers or family.

1 Absent—Definition does not apply.

2 Minimal—Questionable pathology; may be at the upper extreme of normal limits.

3 Mild—One or two clearly formed but infrequent hallucinations, or else a number of vague abnormal perceptions which do not result in distortions of thinking or behavior.

4 Moderate—Hallucinations occur frequently but not continuously, and the patient's thinking and behavior are affected only to a minor extent.

5 Moderate severe—Hallucinations are frequent, may involve more than one sensory modality, and tend to distort thinking and/or disrupt behavior. Patient may have a delusional interpretation of these experiences and respond to them emotionally and, on occasion, verbally as well.

6 Severe—Hallucinations are present almost continuously, causing major disruption of thinking and behavior. Patient treats these as real perceptions, and functioning is impeded by frequent emotional and verbal responses to them.

7 Extreme—Patient is almost totally preoccupied with hallucinations, which virtually dominate thinking and behavior. Hallucinations are provided a rigid delusional interpretation and provoke verbal and behavioral responses, including obedience to command hallucinations.

P4. Excitement: Hyperactivity as reflected in accelerated motor behavior, heightened responsivity to stimuli hypervigilance, or excessive mood lability.

Basis for rating (i.e., rating points 1-7 as herein): Behavioral manifestations during the course of interview, as well as reports of behavior by primary care workers or family.

1 Absent—Definition does not apply.

2 Minimal—Questionable pathology; may be at the upper extreme of normal limits.

3 Mild—Tends to be slightly agitated, hypervigilant, or mildly overaroused throughout the interview, but without distinct episodes of excitement or marked mobility. Speech may be slightly pressured.

4 Moderate—Agitation or overarousal is clearly evident throughout the interview, affecting speech and general mobility, or episodic outbursts occur sporadically.

5 Moderate severe—Significant hyperactivity or frequent outbursts of motor activity are observed, making it difficult for the patient to sit still for longer than several minutes at any given time.

6 Severe—Marked excitement dominates the interview, delimits attention, and to some extent affects personal functions such as eating and sleeping.

7 Extreme—Marked excitement seriously interferes in eating and sleeping and makes interpersonal interactions virtually impossible. Acceleration of speech and motor activity may result in incoherence and exhaustion.

P5. Grandiosity: Exaggerated self-opinion and unrealistic convictions of superiority, including delusions of extraordinary abilities, wealth, knowledge, fame, power, and moral righteousness.

Basis for rating (i.e., rating points 1-7 as herein): Thought content expressed in the interview and its influence on behavior.

1 Absent—Definition does not apply.

2 Minimal—Questionable pathology; may be at the upper extreme of normal limits.

3 Mild—Some expansiveness or boastfulness is evident, but without clear-cut grandiose delusions.

4 Moderate—Feels distinctly and unrealistically superior to others. Some poorly formed delusions about special status or abilities may be present but are not acted upon.

5 Moderate severe—Clear-cut delusions concerning remarkable abilities, status, or power are expressed and influence attitude but not behavior.

6 Severe—Clear-cut delusions of remarkable superiority involving more than one parameter (wealth, knowledge, fame, etc.) are expressed, notably influence interactions, and may be acted upon.

7 Extreme—Thinking, interactions, and behavior are dominated by multiple delusions of amazing ability, wealth knowledge, fame, power, and/or moral stature; which may take on a bizarre quality.

P6. Suspiciousness/persecution: Unrealistic or exaggerated ideas of persecution, as reflected in guardedness, a distrustful attitude, suspicious hypervigilance, or frank delusions that others mean harm.

Basis for rating (i.e., rating points 1-7 as herein): Thought content expressed in the interview and its influence on behavior.

1 Absent—Definition does not apply.

2 Minimal—Questionable pathology; may be at the upper extreme of normal limits.

3 Mild—Presents a guarded or even openly distrustful attitude, but thoughts, interactions, and behavior are minimally affected.

4 Moderate—Distrustfulness is clearly evident and intrudes on the interview and/or behavior, but there is no evidence of persecutory delusions. Alternatively, there may be indication of loosely formed persecutory delusions, but these do not seem to affect the patient's attitude or interpersonal relations.

5 Moderate severe—Patient shows marked distrustfulness, leading to major disruption of interpersonal relations, or else there are clear-cut persecutory delusions that have limited impact on interpersonal relations and behavior.

6 Severe—Clear-cut pervasive delusions of persecution which may be systematized and significantly interfere in interpersonal relations.

7 Extreme—A network of systematized persecutory delusions dominates the patient's thinking, social relations, and behavior.

P7. Hostility: Verbal and nonverbal expressions of anger and resentment, including sarcasm, passive-aggressive behavior, verbal abuse, and assaultiveness.

Basis for rating (i.e., rating points 1-7 as herein): Interpersonal behavior observed during the interview and reports by primary care workers or family.

1 Absent—Definition does not apply.

2 Minimal—Questionable pathology; may be at the upper extreme of normal limits.

3 Mild—Indirect or restrained communication of anger such as sarcasm, disrespect, hostile expressions, and occasional irritability.

4 Moderate—Presents an overtly hostile attitude, showing frequent irritability and direct expression of anger or resentment.

5 Moderate severe—Patient is highly irritable and occasionally verbally abusive or threatening.

6 Severe—Uncooperativeness and verbal abuse or threats notably influence the interview and seriously impact upon social relations. Patient may be violent and destructive but is not physically assaultive toward others.

7 Extreme—Marked anger results in extreme uncooperativeness, precluding other interactions, or in episode(s) of physical assault toward others.

Negative Scale (N)

N1. Blunted affect: Diminished emotional responsiveness as characterized by a reduction in facial expression, modulation of feelings, and communicative gestures.

Basis for rating (i.e., rating points 1-7 as herein): Observation of physical manifestations of affective tone and emotional responsiveness during the course of interview.

1 Absent—Definition does not apply.

2 Minimal—Questionable pathology; may be at the upper extreme of normal limits.

3 Mild—Changes in facial expression and communicative gestures seem to be stilted, forced, artificial, or lacking in modulation.

4 Moderate—Reduced range of facial expression and few expressive gestures result in a dull appearance.

5 Moderate severe—Affect is generally flat, with only occasional changes in facial expression and a paucity of communicative gestures.

6 Severe—Marked flatness and deficiency of emotions exhibited most of the time. There may be unmodulated extreme affective discharges, such as excitement, rage, or inappropriate uncontrolled laughter.

7 Extreme—Changes in facial expression and evidence of communicative gestures are virtually absent. Patient seems constantly to show a barren or “wooden” expression.

N2. Emotional withdrawal: Lack of interest in, involvement with, and affective commitment to life's events.

Basis for rating (i.e., rating points 1-7 as herein): Reports of functioning from primary care workers or family and observation of interpersonal behavior during the course of interview.

1 Absent—Definition does not apply.

2 Minimal—Questionable pathology; may be at the upper extreme of normal limits.

3 Mild—Usually lacks initiative and occasionally may show deficient interest in surrounding events.

4 Moderate—Patient is generally distanced emotionally from the milieu and its challenges but, with encouragement, can be engaged.

5 Moderate severe—Patient is clearly detached emotionally from persons and events in the milieu, resisting all efforts at engagement. Patient appears distant, docile, and purposeless, but can be involved in communication at least briefly and tends to personal needs, sometimes with assistance.

6 Severe—Marked deficiency of interest and emotional commitment results in limited conversation with others and frequent neglect of personal functions, for which the patient requires supervision.

7 Extreme—Patient is almost totally withdrawn, uncommunicative, and neglectful of personal needs as a result of profound lack of interest and emotional commitment.

N3. Poor rapport: Lack of interpersonal empathy, openess in conversation, and sense of closeness, interest, or involvement with the interviewer. This is evidenced by interpersonal distancing and reduced verbal and nonverbal communication.

Basis for rating (i.e., rating points 1-7 as herein): Interpersonal behavior during the course of interview.

1 Absent—Definition does not apply.

2 Minimal—Questionable pathology; may be at the upper extreme of normal limits.

3 Mild—Conversation is characterized by a stilted, strained or artificial tone. It may lack emotional depth or tend to remain on an impersonal, intellectual plane.

4 Moderate—Patient typically is aloof, with interpersonal distance quite evident. Patient may answer questions mechanically, act bored, or express disinterest.

5 Moderate severe—Disinvolvement is obvious and clearly impedes the productivity of the interview. Patient may tend to avoid eye or face contact.

6 Severe—Patient is highly indifferent, with marked interpersonal distance. Answers are perfunctory, and there is little nonverbal evidence of involvement. Eye and face contact are frequently avoided.

7 Extreme—Patient is totally uninvolved with the interviewer. Patient appears to be completely indifferent and consistently avoids verbal and nonverbal interactions during the interview.

N4. Passive/apathetic social withdrawal: Diminished interest and initiative in social interactions due to passivity, apathy, anergy, or avolition. This leads to reduced interpersonal involvement and neglect of activities of daily living.

Basis for rating (i.e., rating points 1-7 as herein): Reports on social behavior from primary care workers or family.

1 Absent—Definition does not apply.

2 Minimal—Questionable pathology; may be at the upper extreme of normal limits.

3 Mild—Shows occasional interest in social activities but poor initiative. Usually engages with others only when approached first by them.

4 Moderate—Passively goes along with most social activities but in a disinterested or mechanical way. Tends to recede into the background.

5 Moderate severe—Passively participates in only a minority of activities and shows virtually no interest or initiative. Generally spends little time with others.

6 Severe—Tends to be apathetic and isolated, participating very rarely in social activities and occasionally neglecting personal needs. Has very few spontaneous social contacts.

7 Extreme—Profoundly apathetic, socially isolated, and personally neglectful.

N5. Difficulty in abstract thinking: Impairment in the use of the abstract-symbolic mode of thinking, as evidenced by difficulty in classification, forming generalizations, and proceeding beyond concrete or egocentric thinking in problem solving tasks.

Basis for rating (i.e., rating points 1-7 as herein): Responses to questions on similarities and proverb interpretation, and use of concrete vs. abstract mode during the course of the interview.

1 Absent—Definition does not apply.

2 Minimal—Questionable pathology; may be at the upper extreme of normal limits.

3 Mild—Tends to give literal or personalized interpretations to the more difficult proverbs and may have some problems with concepts that are fairly abstract or remotely related.

4 Moderate—Often utilizes a concrete mode. Has difficulty with most proverbs and some categories. Tends to be distracted by functional aspects and salient features.

5 Moderate severe—Deals primarily in a concrete mode, exhibiting difficulty with most proverbs and many categories.

6 Severe—Unable to grasp the abstract meaning of any proverbs or figurative expressions and can formulate classifications for only the most simple of similarities. Thinking is either vacuous or locked into functional aspects, salient features, and idiosyncratic interpretations.

7 Extreme—Can use only concrete modes of thinking. Shows no comprehension of proverbs, common metaphors or similes, and simple categories. Even salient and functional attributes do not serve as a basis for classification. This rating may apply to those who cannot interact even minimally with the examiner due to marked cognitive impairment.

N6. Lack of spontaneity and flow of conversation: Reduction in the normal flow of communication associated with apathy, avolition, defensiveness, or cognitive deficit. This is manifested by diminished fluidity and productivity of the verbal-interactional process.

Basis for rating (i.e., rating points 1-7 as herein): Cognitive-verbal processes observed during the course of interview.

1 Absent—Definition does not apply.

2 Minimal—Questionable pathology; may be at the upper extreme of normal limits.

3 Mild—Conversation shows little initiative. Patient's answers tend to be brief and unembellished, requiring direct and leading questions by the interviewer.

4 Moderate—Conversation lacks free flow and appears uneven or halting. Leading questions are frequently needed to elicit adequate responses and proceed with conversation.

5 Moderate severe—Patient shows a marked lack of spontaneity and openness, replying to the interviewer's questions with only one or two brief sentences.

6 Severe—Patient's responses are limited mainly to a few words or short phrases intended to avoid or curtail communication (e g., “I don't know,” “I′m not at liberty to say.”). Conversation is seriously impaired as a result, and the interview is highly unproductive. 7 Extreme—Verbal output is restricted to, at most, an occasional utterance, making conversation not possible.

N7. Stereotyped thinking: Decreased fluidity, spontaneity, and flexibility of thinking, as evidenced in rigid, repetitious, or barren thought content.

Basis for rating (i.e., rating points 1-7 as herein): Cognitive-verbal processes observed during the interview.

1 Absent—Definition does not apply.

2 Minimal—Questionable pathology; may be at the upper extreme of normal limits.

3 Mild—Some rigidity shown in attitudes or beliefs. Patient may refuse to consider alternative positions or have difficulty in shifting from one idea to another.

4 Moderate—Conversation revolves around a recurrent theme, resulting in difficulty in shifting to a new topic.

5 Moderate severe—Thinking is rigid and repetitious to the point that, despite the interviewer's efforts, conversation is limited to only two or three dominating topics.

6 Severe—Uncontrolled repetition of demands, statements, ideas, or questions which severely impairs conversation.

7 Extreme—Thinking, behavior, and conversation are dominated by constant repetition of fixed ideas or limited phrases, leading to gross rigidity, inappropriateness, and restrictiveness of patient's communication.

General Psychopathology Scale (G)

G1. Somatic concern: Physical complaints or beliefs about bodily illness or malfunctions. This may range from a vague sense of ill-being to clear-cut delusions of catastrophic physical disease.

Basis for rating (i.e., rating points 1-7 as herein): Thought content expressed in the interview.

1 Absent—Definition does not apply.

2 Minimal—Questionable pathology; may be at the upper extreme of normal limits.

3 Mild—Distinctly concerned about health or somatic issues, as evidenced by occasional questions and desire for reassurance.

4 Moderate—Complains about poor health or bodily malfunction, but there is no delusional conviction, and overconcern can be allayed by reassurance.

5 Moderate severe—Patient expresses numerous or frequent complaints about physical illness or bodily malfunction, or else patient reveals one or two clear-cut delusions involving these themes but is not preoccupied by them.

6 Severe—Patient is preoccupied by one or a few clear-cut delusions about physical disease or organic malfunction, but affect is not fully immersed in these themes, and thoughts can be diverted by the interviewer with some effort.

7 Extreme—Numerous and frequently reported somatic delusions, or only a few somatic delusions of a catastrophic nature, which totally dominate the patient's affect and thinking.

G2. Anxiety: Subjective experience of nervousness, worry, apprehension, or restlessness, ranging from excessive concern about the present or future to feelings of panic.

Basis for rating (i.e., rating points 1-7 as herein): Verbal report during the course of interview and corresponding physical manifestations.

1 Absent—Definition does not apply.

2 Minimal—Questionable pathology; may be at the upper extreme of normal limits.

3 Mild—Expresses some worry, overconcern, or subjective restlessness, but no somatic and behavioral consequences are reported or evidenced.

4 Moderate—Patient reports distinct symptoms of nervousness, which are reflected in mild physical manifestations such as fine hand tremor and excessive perspiration.

5 Moderate severe—Patient reports serious problems of anxiety which have significant physical and behavioral consequences, such as marked tension, poor concentration, palpitations, or impaired sleep.

6 Severe—Subjective state of almost constant fear associated with phobias, marked restlessness, or numerous somatic manifestations.

7 Extreme—Patient's life is seriously disrupted by anxiety, which is present almost constantly and at times reaches panic proportions or is manifested in actual panic attacks. G3. Guilt feelings: Sense of remorse or self-blame for real or imagined misdeeds in the past.

Basis for rating (i.e., rating points 1-7 as herein): Verbal report of guilt feelings during the course of interview and the influence on attitudes and thoughts.

1 Absent—Definition does not apply.

2 Minimal—Questionable pathology; may be at the upper extreme of normal limits.

3 Mild—Questioning elicits a vague sense of guilt or self-blame for a minor incident, but the patient clearly is not overly concerned.

4 Moderate—Patient expresses distinct concern over his responsibility for a real incident in his life but is not preoccupied with it, and attitude and behavior are essentially unaffected.

5 Moderate severe—Patient expresses a strong sense of guilt associated with self-deprecation or the belief that he deserves punishment. The guilt feelings may have a delusional basis, may be volunteered spontaneously, may be a source of preoccupation and/or depressed mood, and cannot be allayed readily by the interviewer.

6 Severe—Strong ideas of guilt take on a delusional quality and lead to an attitude of hopelessness or worthlessness. The patient believes he should receive harsh sanctions for the misdeeds and may even regard his current life situation as such punishment.

7 Extreme—Patient's life is dominated by unshakable delusions of guilt, for which he feels deserving of drastic punishment, such as life imprisonment, torture, or death. There may be associated suicidal thoughts or attribution of others' problems to one's own past misdeeds.

G4. Tension: Overt physical manifestations of fear, anxiety, and agitation, such as stiffness, tremor, profuse sweating, and restlessness.

Basis for rating (i.e., rating points 1-7 as herein): Verbal report attesting to anxiety and, thereupon, the severity of physical manifestations of tension observed during the interview.

1 Absent—Definition does not apply.

2 Minimal—Questionable pathology; may be at the upper extreme of normal limits.

3 Mild—Posture and movements indicate slight apprehensiveness, such as minor rigidity, occasional restlessness, shifting of position, or fine rapid hand tremor.

4 Moderate—A clearly nervous appearance emerges from various manifestations, such as fidgety behavior, obvious hand tremor, excessive perspiration, or nervous mannerisms.

5 Moderate severe—Pronounced tension is evidenced by numerous manifestations, such as nervous shaking, profuse sweating, and restlessness, but conduct in the interview is not significantly affected.

6 Severe—Pronounced tension to the point that interpersonal interactions are disrupted. The patient for example, may be constantly fidgeting, unable to sit still for long, or show hyperventilation.

7 Extreme—Marked tension is manifested by signs of panic or gross motor acceleration, such as rapid restless pacing and inability to remain seated for longer than a minute, which makes sustained conversation not possible.

G5. Mannerisms and posturing: Unnatural movements or posture as characterized by an awkward, stilted, disorganized, or bizarre appearance.

Basis for rating (i.e., rating points 1-7 as herein): Observation of physical manifestations during the course of interview as well as reports from primary care workers or family.

1 Absent—Definition does not apply.

2 Minimal—Questionable pathology; may be at the upper extreme of normal limits.

3 Mild—Slight awkwardness in movements or minor rigidity of posture.

4 Moderate—Movements are notably awkward or disjointed, or an unnatural posture is maintained for brief periods.

5 Moderate severe—Occasional bizarre rituals or contorted posture are observed, or an abnormal position is sustained for extended periods.

6 Severe—Frequent repetition of bizarre rituals, mannerisms, or stereotyped movements, or a contorted posture is sustained for extended periods..

7 Extreme—Functioning is seriously impaired by virtually constant involvement in ritualistic, manneristic, or stereotyped movements or by an unnatural fixed posture which is sustained most of the time.

G6. Depression: Feelings of sadness, discouragement, helplessness, and pessimism.

Basis for rating (i.e., rating points 1-7 as herein): Verbal report of depressed mood during the course of interview and its observed influence on attitude and behavior.

1 Absent—Definition does not apply.

2 Minimal—Questionable pathology; may be at the upper extreme of normal limits.

3 Mild—Expresses some sadness or discouragement only on questioning. but there is no evidence of depression in general attitude or demeanor.

4 Moderate—Distinct feelings of sadness or hopelessness, which may be spontaneously divulged, but depressed mood has no major impact on behavior or social functioning, and the patient usually can be cheered up.

5 Moderate severe—Distinctly depressed mood is associated with obvious sadness, pessimism, loss of social interest, psychomotor retardation, and some interference in appetite and sleep. The patient cannot be easily cheered up.

6 Severe—Markedly depressed mood is associated with sustained feelings of misery, occasional crying, hopelessness, and worthlessness. In addition, there is major interference in appetite and/or sleep, as well as, in normal motor and social functions, with possible signs of self-neglect.

7 Extreme—Depressive feelings seriously interfere in most major functions. The manifestations include frequent crying, pronounced somatic symptoms, impaired concentration, psychomotor retardation, social disinterest, self-neglect, possible depressive or nihilistic delusions, and/or possible suicidal thoughts or action.

G7. Motor retardation: Reduction in motor activity as reflected in slowing or lessening of movements and speech, diminished responsiveness to stimuli, and reduced body tone.

Basis for rating (i.e., rating points 1-7 as herein): Manifestations during the course of interview as well as reports by primary care workers or family.

1 Absent—Definition does not apply.

2 Minimal—Questionable pathology; may be at the upper extreme of normal limits.

3 Mild—Slight but noticeable diminution in rate of movements and speech. Patient may be somewhat underproductive in conversation and gestures.

4 Moderate—Patient is clearly slow in movements, and speech may be characterized by poor productivity, including long response latency, extended pauses, or slow pace.

5 Moderate severe—A marked reduction in motor activity renders communication highly unproductive or delimits functioning in social and occupational situations. Patient can usually be found sitting or lying down.

6 Severe—Movements are extremely slow, resulting in a minimum of activity and speech. Essentially the day is spent sitting idly or lying down.

7 Extreme—Patient is almost completely immobile and virtually unresponsive to external stimuli.

G8. Uncooperativeness: Active refusal to comply with the will of significant others, including the interviewer, hospital staff, or family, which may be associated with distrust, defensiveness, stubbornness, negativism, rejection of authority, hostility, or belligerence.

Basis for rating (i.e., rating points 1-7 as herein): Interpersonal behavior observed during the course of interview as well as reports by primary care workers or family.

1 Absent—Definition does not apply.

2 Minimal—Questionable pathology; may be at the upper extreme of normal limits.

3 Mild—Complies with an attitude of resentment, impatience, or sarcasm. May inoffensively object to sensitive probing during the interview.

4 Moderate—Occasional outright refusal to comply with normal social demands, such as making own bed, attending scheduled programs, etc. The patient may project a hostile, defensive, or negative attitude but usually can be worked with.

5 Moderate severe—Patient frequently is incompliant with the demands of his milieu and may be characterized by others as an “outcast” or having “a serious attitude problem.” Uncooperativeness is reflected in obvious defensiveness or irritability with the interviewer and possible unwillingness to address many questions.

6 Severe—Patient is highly uncooperative, negativistic, and possibly also belligerent. Refuses to comply with most social demands and may be unwilling to initiate or conclude the full interview.

7 Extreme—Active resistance seriously impacts on virtually all major areas of functioning. Patient may refuse to join in any social activities, tend to personal hygiene, converse with family or staff, and participate even briefly in an interview.

G9. Unusual thought content: Thinking characterized by strange, fantastic, or bizarre ideas, ranging from those which are remote or atypical to those which are distorted, illogical, and patently absurd.

Basis for rating (i.e., rating points 1-7 as herein): Thought content expressed during the course of interview.

1 Absent—Definition does not apply.

2 Minimal—Questionable pathology; may be at the upper extreme of normal limits.

3 Mild—Thought content is somewhat peculiar or idiosyncratic, or familiar ideas are framed in an odd context.

4 Moderate—Ideas are frequently distorted and occasionally seem quite bizarre.

5 Moderate severe—Patient expresses many strange and fantastic thoughts (e.g., being the adopted son of a king, being an escapee from death row) or some which are patently absurd (e.g., having hundreds of children, receiving radio messages from outer space through a tooth filling).

6 Severe—Patient expresses many illogical or absurd ideas or some which have a distinctly bizarre quality (e.g., having three heads, being a visitor from another planet).

7 Extreme—Thinking is replete with absurd, bizarre, and grotesque ideas.

G10. Disorientation: Lack of awareness of one's relationship to the milieu, including persons, place, and time, which may be due to confusion or withdrawal.

Basis for rating (i.e., rating points 1-7 as herein): Responses to interview questions on orientation.

1 Absent—Definition does not apply.

2 Minimal—Questionable pathology; may be at the upper extreme of normal limits.

3 Mild—General orientation is adequate but there is some difficulty with specifics. For example, patient knows his location but not the street address, knows hospital staff names but not their functions, knows the month but confuses the day of week with an adjacent day, or errs in the date by more than two days. There may be narrowing of interest evidenced by familiarity with the immediate but not extended milieu, such as ability to identify staff but not the Mayor, Governor, or President.

4 Moderate—Only partial success in recognizing persons, places, and time. For example, patient knows he is in a hospital but not its name, knows the name of his city but not the borough or district, knows the name of his primary therapist but not many other direct care workers, knows the year and season but not sure of the month.

5 Moderate severe—Considerable failure in recognizing persons, place, and time. Patient has only a vague notion of where he is and seems unfamiliar with most people in his milieu. He may identify the year correctly or nearly so, but not know the current month, day of week, or even the season.

6 Severe—Marked failure in recognizing persons, place, and time. For example, patient has no knowledge of his whereabouts, confuses the date by more than one year, can name only one or two individuals in his current life.

7 Extreme—Patient appears completely disoriented with regard to persons, place, and time. There is gross confusion or total ignorance about one's location, the current year, and even the most familiar people, such as parents, spouse, friends, and primary therapist.

G11. Poor attention: Failure in focused alertness manifested by poor concentration, distractibility from internal and external stimuli, and difficulty in harnessing, sustaining, or shifting focus to new stimuli.

Basis for rating (i.e., rating points 1-7 as herein): Manifestations during the course of interview.

1 Absent—Definition does not apply.

2 Minimal—Questionable pathology; may be at the upper extreme of normal limits.

3 Mild—Limited concentration evidenced by occasional vulnerability, to distraction or faltering attention toward the end of the interview.

4 Moderate—Conversation is affected by the tendency to be easily distracted, difficulty in long sustaining concentration on a given topic, or problems in shifting attention to new topics.

5 Moderate severe—Conversation is seriously hampered by poor concentration, distractibility, and difficulty in shifting focus appropriately.

6 Severe—Patient's attention can be harnessed for only brief moments or with great effort, due to marked distraction by internal or external stimuli.

7 Extreme—Attention is so disrupted that even brief conversation is not possible.

G12. Lack of judgment and insight: Impaired awareness or understanding of one's own psychiatric condition and life situation. This is evidenced by failure to recognize past or present psychiatric illness or symptoms, denial of need for psychiatric hospitalization or treatment, decisions characterized by poor anticipation of consequences, and unrealistic short-term and long-range planning.

Basis for rating (i.e., rating points 1-7 as herein): Thought content expressed during the interview.

1 Absent—Definition does not apply.

2 Minimal—Questionable pathology; may be at the upper extreme of normal limits.

3 Mild—Recognizes having a psychiatric disorder but clearly underestimates its seriousness, the implications for treatment, or the importance of taking measures to avoid relapse. Future planning may be poorly conceived.

4 Moderate—Patient shows only a vague or shallow recognition of illness. There may be fluctuations in acknowledgement of being ill or little awareness of major symptoms which are present, such as delusions, disorganized thinking, suspiciousness, and social withdrawal. The patient may rationalize the need for treatment in terms of its relieving lesser symptoms, such as anxiety, tension, and sleep difficulty.

5 Moderate severe—Acknowledges past but not present psychiatric disorder. If challenged, the patient may concede the presence of some unrelated or insignificant symptoms, which tend to be explained away by gross misinterpretation or delusional thinking. The need for psychiatric treatment similarly goes unrecognized.

6 Severe—Patient denies ever having had a psychiatric disorder. He disavows the presence of any psychiatric symptoms in the past or present and, though compliant, denies the need for treatment and hospitalization.

7 Extreme—Emphatic denial of past and present psychiatric illness. Current hospitalization and treatment are given a delusional interpretation (e.g.. as punishment for misdeeds, as persecution by tormentors, etc.), and the patient may thus refuse to cooperate with therapists, medication, or other aspects of treatment.

G13. Disturbance of volition: Disturbance in the willful initiation, sustenance, and control of one's thoughts, behavior, movements, and speech.

Basis for rating (i.e., rating points 1-7 as herein): Thought content and behavior manifested in the course of interview.

1 Absent—Definition does not apply.

2 Minimal—Questionable pathology; may be at the upper extreme of normal limits.

3 Mild—There is evidence of some indecisiveness in conversation and thinking, which may impede verbal and cognitive processes to a minor extent.

4 Moderate—Patient is often ambivalent and shows clear difficulty in reaching decisions.

Conversation may be marred by alternation in thinking, and in consequence verbal and cognitive functioning are clearly impaired.

5 Moderate severe—Disturbance of volition interferes in thinking as well as behavior. Patient shows pronounced indecision that impedes the initiation and continuation of social and motor activities, which also may be evidenced in halting speech.

6 Severe—Disturbance of volition interferes in the execution of simple, automatic motor functions, such as dressing and grooming, and markedly affects speech.

7 Extreme—Almost complete failure of volition is manifested by gross inhibition of movement and speech, resulting in immobility and/or mutism.

G14. Poor impulse control: Disordered regulation and control of action on inner urges resulting in sudden, unmodulated, arbitrary, or misdirected discharge of tension and emotions without concern about consequences.

Basis for rating (i.e., rating points 1-7 as herein): Behavior during the course of interview and reported by primary care workers or family.

1 Absent—Definition does not apply.

2 Minimal—Questionable pathology; may be at the upper extreme of normal limits.

3 Mild—Patient tends to be easily angered and frustrated when facing stress or denied gratification but rarely acts on impulse.

4 Moderate—Patient gets angered and verbally abusive with minimal provocation. May be occasionally threatening, destructive, or have one or two episodes involving physical confrontation or a minor brawl.

5 Moderate severe—Patient exhibits repeated impulsive episodes involving verbal abuse, destruction of property, or physical threats. There may be one or two episodes involving serious assault, for which the patient requires isolation, physical restraint, or p.r.n. sedation.

6 Severe—Patient frequently is impulsively aggressive, threatening, demanding, and destructive, without any apparent consideration of consequences. Shows assaultive behavior and may also be sexually offensive and possibly respond behaviorally to hallucinatory commands.

7 Extreme—Patient exhibits homicidal attacks, sexual assaults, repeated brutality, or self-destructive behavior. Requires constant direct supervision or external constraints because of inability to control dangerous impulses.

G15. Preoccupation: Absorption with internally generated thoughts and feelings and with autistic experiences to the detriment of reality orientation and adaptive behavior.

Basis for rating (i.e., rating points 1-7 as herein): Interpersonal behavior observed during the course of interview.

1 Absent—Definition does not apply.

2 Minimal—Questionable pathology; may be at the upper extreme of normal limits.

3 Mild—Excessive involvement with personal needs or problems, such that conversation veers back to egocentric themes and there is diminished concern exhibited toward others.

4 Moderate—Patient occasionally appears self-absorbed, as if daydreaming or involved with internal experiences, which interferes with communication to minor extent.

5 Moderate severe—Patient often appears to be engaged in autistic experiences, as evidenced by behaviors that significantly intrude on social and communicational functions, such as the presence of a vacant stare, muttering or talking to oneself, or involvement with stereotyped motor patterns.

6 Severe—Marked preoccupation with autistic experiences, which seriously delimits concentration, ability to converse, and orientation to the milieu. The patient frequently may be observed smiling, laughing, muttering, talking, or shouting to himself.

7 Extreme—Gross absorption with autistic experiences, which profoundly affects all major realms of behavior. The patient constantly may be responding verbally and behaviorally to hallucinations and show little awareness of other people or the external milieu.

G16. Active social avoidance: Diminished social involvement associated with unwarranted fear, hostility, or distrust.

Basis for rating (i.e., rating points 1-7 as herein): Reports of social functioning by primary care workers or family.

1 Absent—Definition does not apply.

2 Minimal—Questionable pathology; may be at the upper extreme of normal limits.

3 Mild—Patient seems ill at ease in the presence of others and prefers to spend time alone, although he participates in social functions when required.

4 Moderate—Patient begrudgingly attends all or most social activities but may need to be persuaded or may terminate prematurely on account of anxiety, suspiciousness, or hostility.

5 Moderate severe—Patient fearfully or angrily keeps away from many social interactions despite others' efforts to engage him. Tends to spend unstructured time alone.

6 Severe—Patient participates in very few social activities because of fear, hostility, or distrust. When approached, the patient shows a strong tendency to break off interactions, and generally he tends to isolate himself from others.

7 Extreme—Patient cannot be engaged in social activities because of pronounced fears, hostility, or persecutory delusions. To the extent possible, he avoids all interactions and remains isolated from others.

The term “positive symptoms associated with schizophrenia”, as used herein, refers to each of the items (i.e., P1 to P7) of the Positive Scale (P), according to the PANSS scale, as defined herein. In one embodiment, he term “positive symptoms associated with schizophrenia”, as used herein, refers to items selected from the group comprising P1, P2, P3, P4, P5, P6 and P7, of the Negative Scale (N), according to the PANSS scale, as defined herein. The term “delusions associated with schizophrenia”, as used herein, refers to item P1 of the Positive

Scale (P), according to the PANSS scale, as defined herein. The term “disorganized thinking” associated with schizophrenia, as used herein, refers to item P2 of the Positive Scale (P), according to the PANSS scale, as defined herein. The term “hallucinations associated with schizophrenia”, as used herein, refers to item P3 of the Positive Scale (P), according to the PANSS scale, as defined herein. The term “excitement associated with schizophrenia”, as used herein, refers to item P4 of the Positive Scale (P), according to the PANSS scale, as defined herein. The term “grandiosity associated with schizophrenia”, as used herein, refers to item P5 of the Positive Scale (P), according to the PANSS scale, as defined herein. The term “suspiciousness associated with schizophrenia as used herein, refers to item P6 of the Positive Scale (P), according to the PANSS scale, as defined herein. The term “hostility associated with schizophrenia”, as used herein, refers to item P7 of the Positive Scale (P), according to the PANSS scale, as defined herein.

The term “negative symptoms associated with schizophrenia”, as used herein, refers to each of the items (i.e., N1 to N7) of the Negative Scale (N), according to the PANSS scale, as defined herein. In one embodiment, he term “negative symptoms associated with schizophrenia”, as used herein, refers to items selected from the group comprising N1, N2, N3, N4, N5, N6 and N7, of the Negative Scale (N), according to the PANSS scale, as defined herein. The term “blunted affect associated with schizophrenia”, as used herein, refers to item N1 of the Negative Scale (N), according to the PANSS scale, as defined herein. The term “emotional withdrawal associated with schizophrenia”, as used herein, refers to item N2 of the Negative Scale (N), according to the PANSS scale, as defined herein. The term “poor rapport associated with schizophrenia”, as used herein, refers to item N3 of the Negative Scale (N), according to the PANSS scale, as defined herein. The term “apathetic social withdrawal associated with schizophrenia”, as used herein, refers to item N4 of the Negative Scale (N), according to the PANSS scale, as defined herein. The term “difficulty in abstract thinking associated with schizophrenia”, as used herein, refers to item N5 of the Negative Scale (N), according to the PANSS scale, as defined herein. The term “lack of spontaneity and flow of conversation associated with schizophrenia” as used herein, refers to item N6 of the Negative Scale (N), according to the PANSS scale, as defined herein. The term “stereotyped thinking associated with schizophrenia”, as used herein, refers to item N7 of the Negative Scale (N), according to the PANSS scale, as defined herein.

The term “depression”, as used herein, is defined, for example, with reference to the Beck Depression Inventory Second Edition (BDI-II) [Beck, A.T., Brown, G. K. (1996), Manual for the Beck Depression Inventory-II, San Antonio, TX: Psychological Corporation, which is incorporated herein by reference]. The Beck Depression Inventory Second Edition (BDI-II) is a 21-item self-report instrument for measuring the severity of depression. It provides a total score index of current depression symptom severity [i.e., minimal depression (0-13), mild depression (14-19), moderate depression (20-28), severe depression (29-63)].

The term “depression associated with schizophrenia”, as used herein, refers to a schizophrenic patient with depression, as defined herein, for example with moderate to severe depression, as assessed by a BDI-II score of >19. In one embodiment, “depression associated with schizophrenia”, as used herein, refers to moderate depression associated with schizophrenia or severe depression associated with schizophrenia, as assessed by BDI-II.

The term “insomnia”, as used herein, refers, without limitation, to difficulty in initiating sleep, difficulty in maintaining sleep, waking up earlier than desired or having difficulty sleeping without intervention. For example, insomnia may be assessed (e.g., by % improvement in score relative to baseline) from the Insomnia Severity Index (ISI).

The ISI is a 7-item instrument measure that provides a total score of insomnia severity [i.e., total score categories: 0-7=no clinically significant insomnia (absence of insomnia), 8-14=subthreshold of insomnia, 15-21=clinical insomnia (moderate insomnia), 22-28=clinical insomnia (severe insomnia); e.g., in Sleep. 2011; 34(5):601-608].

The Motivation and Pleasure Scale-Self Report (MAP-SR) is a 15-item self-report measure that provides a total score index of current motivation/pleasure negative symptoms [Compr Psychiatry. 2013; 54(5), 568-74, which is incorporated herein by reference].

The World Health Organization Quality of Life (WHOQOL-BREF) scale is a 26-item clinician-administered structured interview that assesses psychological functioning and quality of life in four primary domains: social relationships, psychological, physical, and environment [e.g., WHOQOL-BREF, Introduction, administration, Scorimng and Generic Version of the Assessment, Field Trial Version, Dec. 1996, World Health Organization; available at www.who.int/mental health/media/en/76.pdf; Psychol Med 1998, 28(3), 551-558, which is incorporated herein by reference].

The InterSePT Scale for Suicidal Thinking-Plus (ISST-Plus) is an instrument for the assessment of current suicidal ideation in patients with schizophrenia [e.g., Innov Clin Neurosci., 2014, 11(9-10), 32-46, which is incorporated herein by reference].

The Brief Medication Questionnaire (BMQ) is a self-report of medication usage, including what medications the patient is currently taking, how they took each medication in the past week, drug effects and bothersome features, and difficulties remembering to take their medication (i.e., a tool for screening medication adherence and barriers to adherence; e.g., in Patient Educ Couns., 1999, 37(2), 113-124, which is incorporated herein by reference].

The term “antipsychotic”, as used herein, refers to a neuroleptic drug used to treat a psychotic disorder, such as schizophrenia. In one embodiment, the antipsychotic is, for example, selected from the group comprising a typical antipsychotic and an atypical antipsychotic. In another embodiment, the antipsychotic is a typical antipsychotic. In yet another embodiment, the antipsychotic is an atypical antipsychotic.

The term “typical antipsychotic”, as used herein, refers to a first-generation antipsychotic, for example selected from the group comprising a butyrophenone (e.g., haloperidol), a diphenylbutylpiperidine (e.g., pimozide), a phenothiazine (e.g., chlorpromazine, fluphenazine, perphenazine, prochlorperazine, trifluoperazine), and a thioxanthene (e.g., thiothixene). In one embodiment, the typical antipsychotic is selected from the group comprising haloperidol, pimozide, chlorpromazine, fluphenazine, perphenazine, prochlorperazine, trifluoperazine, and thiothixene; or salts thereof.

The term “atypical antipsychotic”, as used herein, refers to a second-generation antipsychotic, for example selected from the group comprising a benzamide (e.g., sultopride), a benzisoxazole/benzisothiazole (e.g., lurasidone, paliperidone, risperidone), a phenylpiperazine/quinolinone (e.g., aripiprazole, brexpiprazole, cariprazine) a tricyclic (e.g., clozapine, olanzapine, quetiapine, asenapine, zotepine). In one embodiment, the atypical antipsychotic is selected from the group comprising sultopride, lurasidone, paliperidone, risperidone, brexpiprazole, cariprazine, clozapine, olanzapine, quetiapine, asenapine and zotepine; or salts thereof.

The term “antidepressant”, as used herein, refers to a medication commonly used to treat depression, such as a serotonin reuptake inhibitor (SSRI, e.g., fluoxetine, citalopram, sertraline, paroxetine, escitalopram, fluvoxamine, vilazodone, vortioxetine), a serotonin and norepinephrine reuptake inhibitor (SNRI, e.g., venlafaxine, duloxetine, desvenlafaxine, milnacipran, levomilnacipran), bupropion, a tricyclic antidepressant (e.g., amitriptyline, nortriptyline, doxepin, desipramine, imipramine, protriptyline, trimipramine, clomipramine), a tetracyclic antidepressant (e.g., maprotiline, mianserin, mirtazapine, setiptiline), or a monoamine oxidase inhibitor (MAOI, e.g., isocarboxazid, phenelzine, selegiline, tranylcypromine). In one embodiment, the antidepressant is selected from the group comprising a serotonin reuptake inhibitor (SSRI, e.g., fluoxetine, citalopram, sertraline, paroxetine, escitalopram, fluvoxamine, vilazodone, vortioxetine), a serotonin and norepinephrine reuptake inhibitor (SNRI, e.g., venlafaxine, duloxetine, desvenlafaxine, milnacipran, levomilnacipran), bupropion, a tricyclic antidepressant (e.g., amitriptyline, nortriptyline, doxepin, desipramine, imipramine, protriptyline, trimipramine, clomipramine), a tetracyclic antidepressant (e.g., maprotiline, mianserin, mirtazapine, setiptiline), and a monoamine oxidase inhibitor (MAOI, e.g., isocarboxazid, phenelzine, selegiline, tranylcypromine). In another embodiment, the antidepressant is selected from the group comprising fluoxetine, citalopram, sertraline, paroxetine, escitalopram, fluvoxamine, vilazodone, vortioxetine, venlafaxine, duloxetine, desvenlafaxine, milnacipran, levomilnacipran, bupropion, amitriptyline, nortriptyline, doxepin, desipramine, imipramine, protriptyline, trimipramine, clomipramine, maprotiline, mianserin, mirtazapine, setiptiline, isocarboxazid, phenelzine, selegiline and tranylcypromine; or salts thereof.

The term “anxiolytic”, as used herein, refers to a drug that inhibits anxiety, such as benzodiazepines (e.g., alprazolam, bromazepam, chlordiazepoxide, clonazepam, clorazepate, diazepam, flurazepam, lorazepam, oxazepam, temazepam, triazolam) or antihistamines (e.g., hydroxyzine). In one embodiment, the anxiolytic is selected from the group comprising alprazolam, bromazepam, chlordiazepoxide, clonazepam, clorazepate, diazepam, flurazepam, lorazepam, oxazepam, temazepam, triazolam, and hydroxyzine; or slats thereof.

The term “treat” “treating” “treatment” or “therapy”, as used herein, means obtaining beneficial or desired results, for example, clinical results. Beneficial or desired results can include, but are not limited to, alleviation of one or more symptoms of schizophrenia, as defined herein, such as positive symptoms of schizophrenia or negative symptoms of schizophrenia, as herein defined. One aspect of the treatment is, for example, that said treatment should have a minimal adverse effect on the patient, e.g., it should have a high level of safety. The term “alleviation”, for example in reference to a symptom of a condition, as used herein, refers to reducing at least one of the frequency and amplitude of a symptom of a condition in a patient. In one embodiment, the term “method for the treatment”, as used herein, refers to “method to treat”.

The term “concomitant”, as used herein, refers to both simultaneous and sequential administration.
As used herein, the term “subject” refers to a mammalian organism, preferably a human being (male or female).
As used herein, the term “patient” refers to a subject who is diseased and would benefit from the treatment.
As used herein, a subject is “in need of” a treatment if such subject (patient) would benefit biologically, medically or in quality of life from such treatment.
The term “pharmaceutical composition” is defined herein to refer to a mixture or solution containing at least one active ingredient or therapeutic agent to be administered to a subject, in order to treat a particular condition (i.e., disease, disorder or condition or at least one of the clinical symptoms thereof) affecting the subject.
As used herein, the term “pharmaceutically acceptable excipient” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 22^{nd} Ed. Mack Printing Company, 2013, pp. 1049-1070). Except insofar as any conventional carrier is incompatible with the active ingredient, its use in the therapeutic or pharmaceutical compositions is contemplated.
The terms “drug”, “active substance”, “active ingredient”, “pharmaceutically active ingredient”, “active agent” or “therapeutic agent” are to be understood as meaning a compound in free form or in the form of a pharmaceutically acceptable salt, in particular compounds of the type specified herein. In particular, reference to antipsychotic, in combination with a further active agent, as used herein (e.g., in any of embodiments herein above, or in any of the claims, herein below), refers to an antipsychotic (e.g., in free form or in a pharmaceutical salt form) in combination with at least one further active agent, for example selected from the group comprising an antidepressant (e.g., in free form or in a pharmaceutical salt form) and an anxiolytic (e.g., in free form or in a pharmaceutical salt form).

The term “cognitive behavioral therapy for schizophrenia”, as used herein, refers to cognitive and behavioral techniques for the treatment of schizophrenia, such as cognitive and behavioral techniques that support management of schizophrenia symptoms (e.g., in The Journal of Nervous and Metal Disease, 2001, 189 (5), 278-287). In one embodiment, cognitive and behavioral techniques that support management of schizophrenia symptoms are based on the cognitive model of the positive symptoms of psychosis (e.g., in Psychol Med., 2001, 31, 189-195). In another embodiment, cognitive and behavioral techniques that support management of schizophrenia symptoms are based on the stress-vulnerability model of schizophrenia (e.g., in Schizophr Bull., 1986, 12, 631-647; in J. Abnorm Psychol. 1977, 86, 103-126). In yet another embodiment, cognitive and behavioral techniques that support management of schizophrenia symptoms are based on the cognitive model of the positive symptoms of psychosis and on the stress-vulnerability model of schizophrenia.

The term “behavioral therapy”, as used herein, refers to, but is not limited to, cognitive behavioral therapy, in particular focused on sleep hygiene, medication adherence, social skills, symptoms of psychosis [i.e., hallucinations, such as auditory hallucinations, delusions, or both] or mood management. In one embodiment, term “behavioral therapy”, as used herein, refers to cognitive behavioral therapy focused on, one or more of, sleep hygiene, medication adherence, social skills, symptoms of psychosis [i.e., hallucinations, such as auditory hallucinations, delusions, or both] and mood management. In another embodiment, term “behavioral therapy”, as used herein, refers to cognitive behavioral therapy focused on sleep hygiene. In yet another embodiment, term “behavioral therapy”, as used herein, refers to cognitive behavioral therapy focused on medication adherence. In still another embodiment, term “behavioral therapy”, as used herein, refers to cognitive behavioral therapy focused on social skills. In a further embodiment, term “behavioral therapy”, as used herein, refers to cognitive behavioral therapy focused on symptoms of psychosis [i.e., hallucinations, such as auditory hallucinations, delusions, or both]. In another embodiment, term “behavioral therapy”, as used herein, refers to cognitive behavioral therapy focused on mood management.

The term “computerized behavioral therapy”, as used herein, refers to the delivery of CBT via an interactive computer interface (e.g., delivered by a smartphone). In some embodiments, “computerized behavioral therapy ” refers to, instead of a face-to-face with a human therapist, a computer-based delivery of CBT without therapist.

The term “medication adherence”, as used herein, refers to the ability to take medications as prescribed (i.e., the degree to which a patient takes medication in accordance with the agreed recommendations from a health care provider).

The term “social skills”, as used herein, refers to the ability to communicate with other people in a manner appropriate (i.e., in a manner that does not violate social and relational norms) and effective (i.e., in a manner that the communication achieves patient's goals in social situations). Social skills training in schizophrenia are, for example, described in Activitas Nervoisa Superior Rediviva, Vol. 54, 2012, 159-170. In one embodiment, social skills comprise, for example, confidence in social situations, mindfulness, exercise and healthy living and goal setting & productivity.

The term “sleep hygiene”, as used herein, refers to sleep practices and habits, for example, by following routines (e.g., a set of rules), that promote nighttime sleep quality and daytime alertness (e.g., by having a regular bedtime, by limiting daytime naps to 30 minutes).

The term “mood management”, as used herein, refers to building skills to effectively manage difficult emotions such as, for example, anger (i.e., anger management), anxiety (i.e., anxiety management) or depression.

The term “symptoms of psychosis”, as used herein, refers to delusions, hallucinations (e.g., auditory hallucinations), as defined herein, or both [e.g., in Continuum (Minneap Minn.). 2015 June; 21(3 Behavioral Neurology and Neuropsychiatry): 715-736].

The term “relapse”, as used herein, refers to, for example, to a return or worsening of schizophrenia symptoms (e.g., positive symptoms, negative symptoms, as defined herein) after a period of remission.

The term “digital-therapeutic”, as used herein, is to be understood in the context of “software as a medical device” (SaMD), for example, as defined by the International Medical Device Regulation Forum (i.e., IMDRF SaMD Working Group in “Software as a Medical Device (SaMD): Key Definitions, 9 December 2013). In one embodiment, SaMD refers to software intended to be used for one or more medical purposes that perform these purpose without being part of a hardware medical device (i.e., its intended purpose is not to drive a hardware medical device).

In one embodiment, SaMD provides means and suggestions for mitigation of a disease. In a further embodiment, SaMD, as used herein, refers to software intended to be used for one or more medical purposes, that perform these purpose without being part of a hardware medical device, and it provides means and suggestions for mitigation of a disease (i.e., provides therapy tools, for example psychosocial tools, such as behavioral therapy, for example cognitive behavioral therapy, for example, by providing skill building to aid treatment e.g., to aid copying with disease's symptoms or prevent relapse). The term “digital-therapeutic application”, as used herein, refers to a software as a medical device (i.e., a digital-therapeutic), as defined herein. In one embodiment, the term “digital-therapeutic”, as used herein, refers to a digiceutical or a digital-therapeutic, or a digital therapy, and thus these terms are interchangeable herein above and below.

The term “computer-implemented” in the expression “computer-implemented behavioral therapy”, as used herein, refers to behavioral therapy implemented by the use of electronic tools such as an online tool, a mobile device (e.g., device 100) or a software application.

The term “computer-implemented mobile device”, as used herein, refers to a portable device (e.g., device 100) that can provide computerized therapy, such as computerized behavioral therapy, in particular computerized cognitive behavioral therapy.

The term “on demand”, as used herein, refers to the ability to allow a user (e.g., a patient) to initiate a computer-implemented mobile device (e.g., a computerized behavioral therapy device), or digital therapeutic, at any desired time.

The term “system-prompted”, as used herein, refers to the ability, for example, of a computer-implemented mobile device (e.g., computerized behavioral therapy device) to prompt a user (e.g., patient) to initiate the therapy provided by such a device at any time, for example once a day.

The term “combination”, as used herein, in relation to an active ingredient (e.g., an antipsychotic) and a computer-implemented mobile device or a software as medical device (i.e., digital-therapeutic), refers to a non-fixed combination or a kit of parts for the combined treatment, wherein the active ingredient [e.g., antipsychotic, and optionally one or more drug partner (e.g., another drug as specified herein, also referred to as further “pharmaceutical active ingredient”, “therapeutic agent” or “co-agent”, such as an antidepressant or an anxiolytic) as a “fixed pharmaceutical combination” or as a “non-fixed pharmaceutical combination”], may be administered independently at the same time or separately within time intervals, especially where these time intervals allow a cooperative effect, e.g., synergistic effect. The terms “co-administration” or “combined administration” or the like as utilized herein are meant to encompass administration of the selected combination to a single subject in need thereof (e.g., a patient).

The term “pharmaceutical combination” refers to either a “fixed pharmaceutical combination” in one unit dosage form (e.g., capsule, tablet, caplets or particulates), a “non-fixed pharmaceutical combination”, or a kit of parts for the combined administration where the antipsychotic and one or more drug partner (e.g., another drug as specified herein, also referred to as further “pharmaceutical active ingredient”, “therapeutic agent”, “combination partner” or “co-agent”) may be administered independently at the same time or separately within time intervals. The term “fixed pharmaceutical combination” means that the active ingredients, e.g., the antipsychotic and one or more combination partners, are both administered to a patient simultaneously in the form of a single entity or dosage. The term “non-fixed pharmaceutical combination” means that the active ingredients, e.g., antipsychotic and one or more combination partners, are both administered to a patient as separate entities either simultaneously or sequentially with no specific time limits, wherein such administration provides therapeutically effective levels of the two compounds in the body of the patient.

As used herein, the terms “free form” or “free forms” refers to the compound in non-salt form, such as the base free form or the acid free form of a respective compound, e.g., the compounds specified herein.

As used herein, the terms “salt”, “salts” or “salt form” refers to an acid addition or base addition salt of a respective compound, e.g., the compounds specified herein. “Salts” include in particular “pharmaceutically acceptable salts”. The term “pharmaceutically acceptable salts” refers to salts that retain the biological effectiveness and properties of the compounds and, which typically are not biologically or otherwise undesirable. The compounds, as specified herein, may be capable of forming acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Pharmaceutically acceptable salts can be synthesized from a basic or acidic moiety, by conventional chemical methods. Generally, such salts can be prepared by reacting the free acid forms of the compound with a stoichiometric amount of the appropriate base (such as Na, Ca, Mg, or K hydroxide, carbonate, bicarbonate or the like), or by reacting the free base form of the compound with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 22^{nd} edition, Mack Publishing Company (2013); and in “Handbook of Pharmaceutical Salts: Properties, Selection, and Use” by Stahl and Wermuth (Wiley-VCH, Weinheim, 2011, 2^{nd} edition).

The compounds specified herein can be administered by conventional route, in particular orally, such as in the form of tablets, capsules, caplets or particulates, which can be manufactured according to pharmaceutical techniques as known in the art (for example in “Remington Essentials of Pharmaceutics, 2013, 1^{st} Edition, edited by Linda Felton, published by Pharmaceutical Press 2012, ISBN 978 0 85711 105 0; in particular Chapter 30), wherein pharmaceutical excipients are, for example, as described in “Handbook of Pharmaceutical Excipients, 2012, 7^{th} Edition, edited by Raymond C. Rowe, Paul J. Sheskey, Walter G. Cook and Marian E. Fenton, ISBN 978 0 85711 027 5”.

The pharmaceutical composition or combination of the present invention can be in a unit dosage form (e.g., tablet, capsule, caplet or particulate), wherein the appropriate dosage of the active ingredient may vary depending upon a variety of factors, such as, for example, the age, weight, sex, the route of administration or salt employed.

### EXAMPLES

The following Examples serve to illustrate this disclosure without limiting the scope thereof.

### Example 1

### Digital-Therapeutic Application and User Interfaces

### Computing Systems

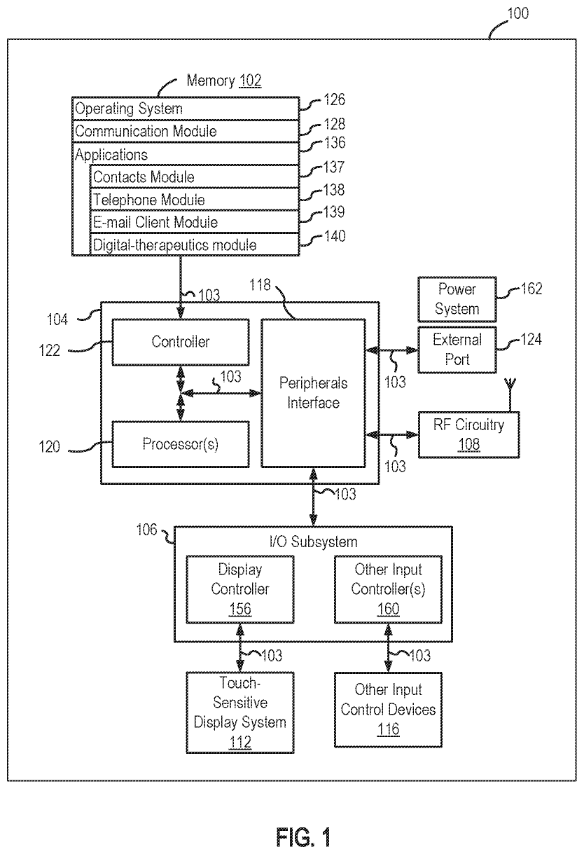

FIG. 1 is a block diagram illustrating an exemplary portable multifunction device 100 with touch-sensitive display system 112 for performing at least some of the methods discussed herein, including methods for providing targeted deliver of digital therapies. Touch-sensitive display 112 is sometimes called a “touch screen” for convenience and is sometimes known as or called a “touch-sensitive display system.” Device 100 includes memory 102, Display controller 156, input/output (I/O) subsystem 106, other input control devices 116, other input controller(s) 160, external port 124, memory controller 122, one or more processing units (e.g., CPU(s), GPU(s), or other suitable processing units known in the art) 120, peripherals interface 118, power system 162, and RF circuitry 108. These components optionally communicate over one or more communication buses or signal lines 103. Device 100 is optionally in communication with one or more external electronic devices (e.g., via RF circuitry 108).

It should be appreciated that device 100 is only one example of a portable multifunction device, and that device 100 optionally has more or fewer components than shown, optionally combines two or more components, or optionally has a different configuration or arrangement of the components. The various components shown in FIG. 1 are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

In some embodiments, the software components stored in memory 102 include operating system 126, communication module (or set of instructions) 128, and applications (or sets of instructions) 136. Operating system 126 (e.g., Darwin®, RTXC®, LINUX®, UNIX®, OS X®, iOS®, ANDROID®, WINDOWS®, or an embedded operating system such as VxWorks®) includes various software components and/or drivers for controlling and managing general system tasks (e.g., memory management, storage device control, power management, etc.) and facilitates communication between various hardware and software components. Communication module 128 facilitates communication with other devices over one or more external ports 124 and also includes various software components for handling data received by RF circuitry 108 and/or external port 124. External port 124 (e.g., Universal Serial Bus (USB), FIREWIRE, etc.) is adapted for coupling directly to other devices or indirectly over a network (e.g., the Internet, wireless LAN, etc.).

Applications 136 optionally include the following modules (or sets of instructions), or a subset or superset thereof:
Contacts module 137;
E-mail client module 139;
Telephone module 138; and
Digital-therapeutics module (also referred to herein as “digital-therapeutics application”) 140.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs, procedures, or modules, and thus various subsets of these modules are, optionally, combined or otherwise rearranged in various embodiments.

I/O subsystem 106 couples input/output peripherals on device 100, such as touch screen 112 and other input control devices 116, to peripherals interface 118.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wde Web (WWW), an intranet and/or a wireless network, such as a cellular telephone network, a wireless local area network (LAN) and/or a metropolitan area network (MAN), and other devices by wireless communication.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Peripherals interface 118 can be used to couple input and output peripherals of the device to CPU 120 and memory 102. The one or more processors 120 run or execute various software programs and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

In some embodiments, device 100 is a mobile device such as a small, handheld computing device that can run applications software. Examples, not limited to, include smartphones and mobile computers (e.g., tablet computer, wearable computer). In some embodiments, a smartphone is a mobile phone having an operating system and computing capability, further includes applications software. Examples of smartphones include, but are not limited to, iPhones and Android smart devices. In some embodiments, a tablet computer includes a mobile computer with display, circuitry and battery in a single housing, that typically, comprises a touchscreen with finger or stylus gestures substituting for the use of computer mouse and keyboard. Examples of tablet computers include, but are not limited to, Apple iPad tablets and Samsung Galaxy Tab tablets. In some embodiments, a wearable computer is a smartwatch (e.g., Apple Watch, Samsung Gear smartwatch, LG G Watch, and Sony SmartWatch) and/or computerized wristbands (i.e., smart wristband).

In some embodiments, device 100 is a laptop computer (e.g., a portable computer designed to be carried by an individual, typically having integrated display, integrated hardware keyboard, and one or more integrated touch sensitive surfaces.

User Interfaces

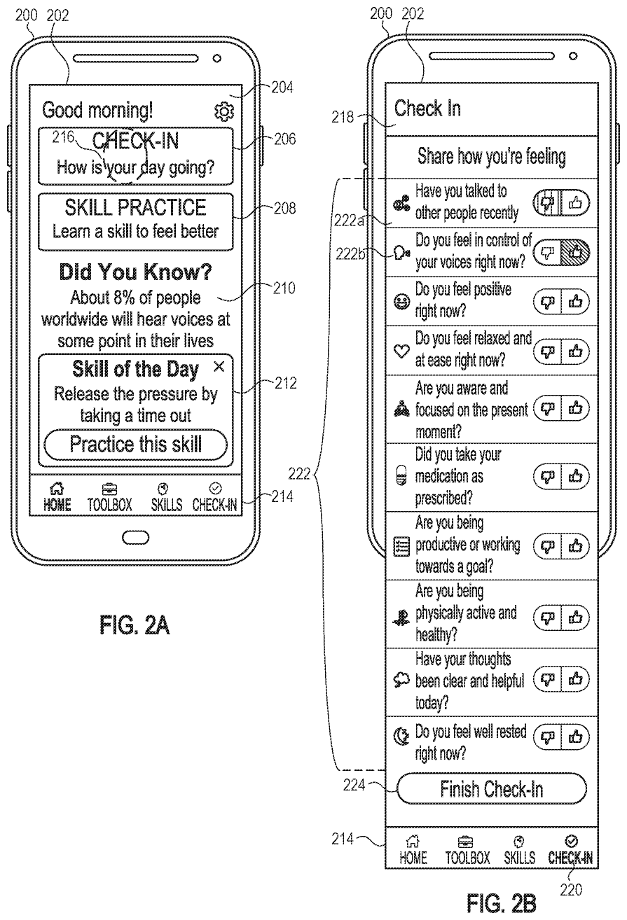

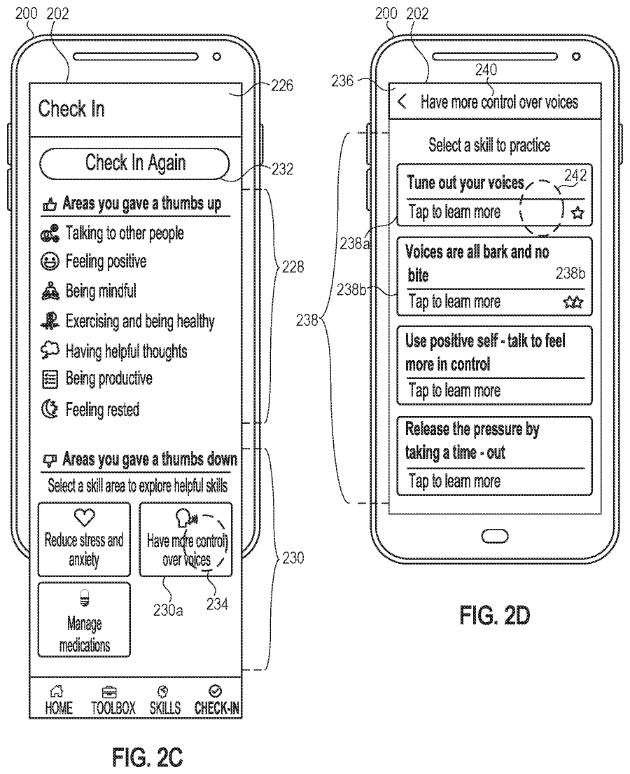

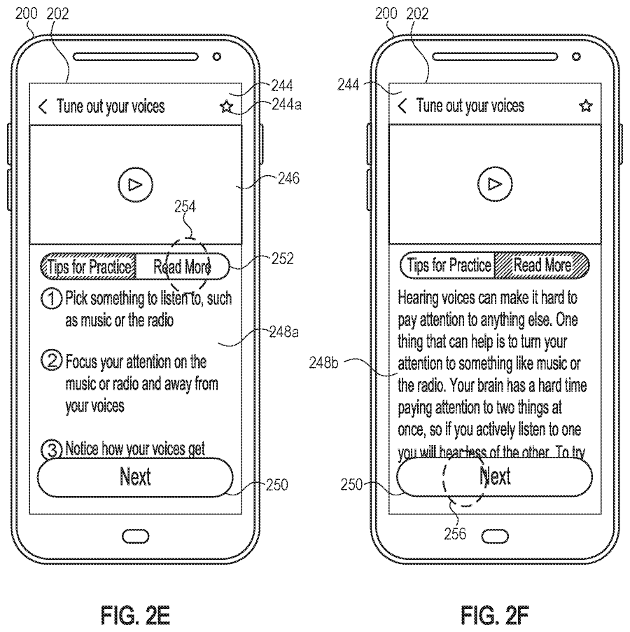

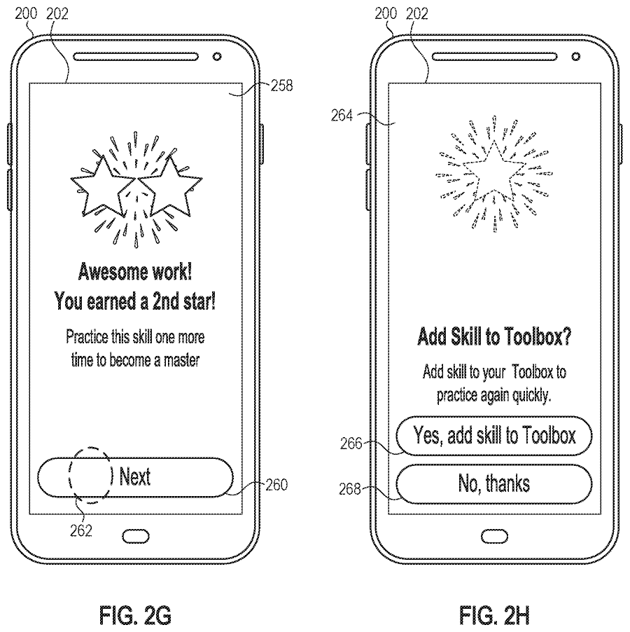

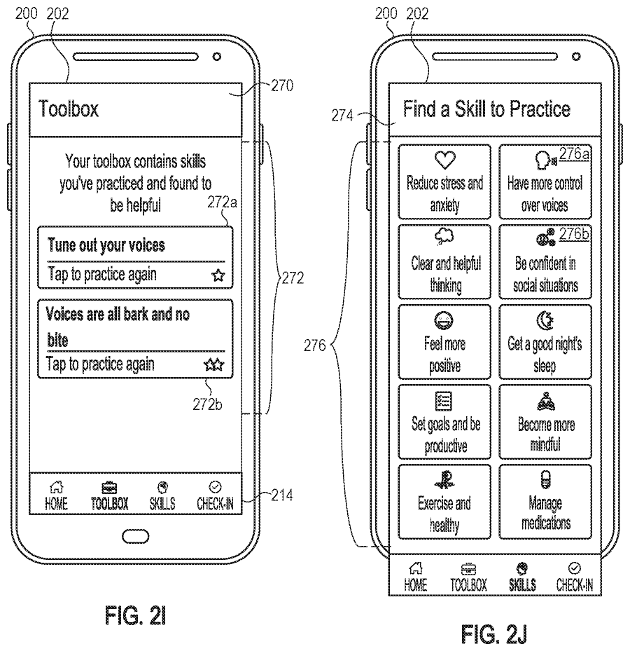

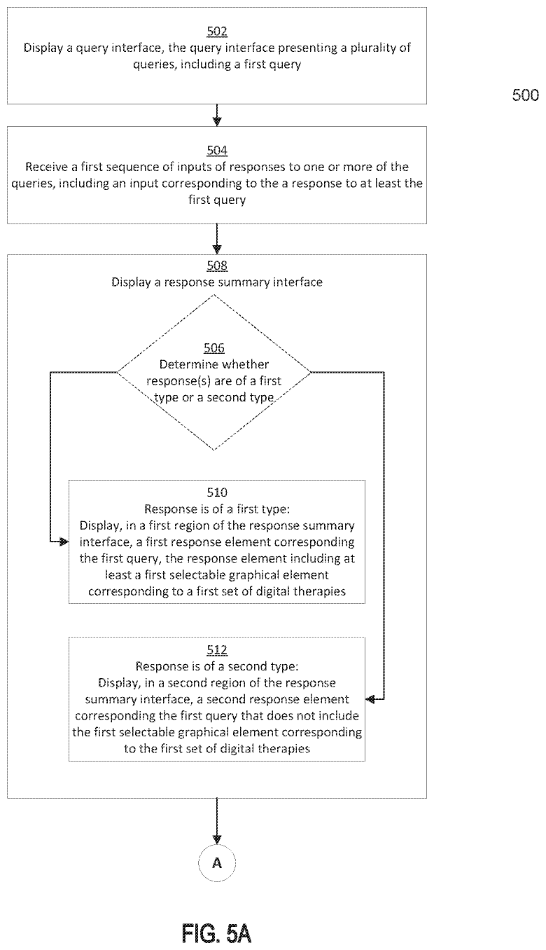

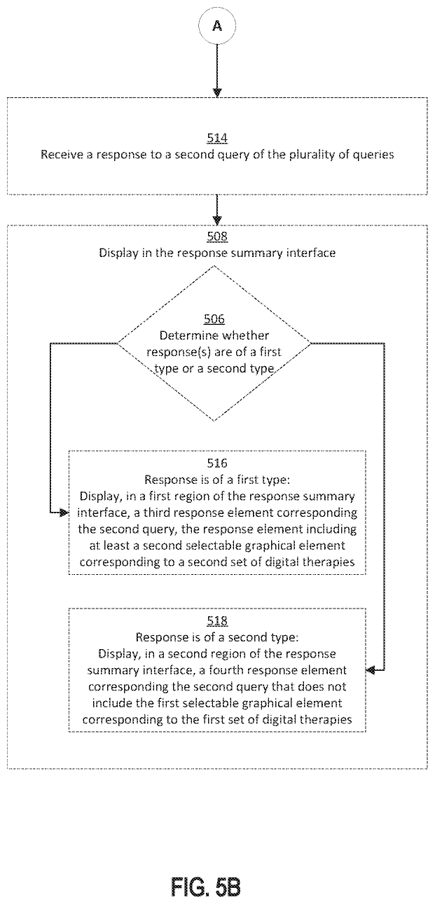

FIGS. 2A-2J illustrate exemplary user interfaces for providing targeted delivery of digital therapies, in accordance with some examples. The user interfaces in these figures are used to illustrate the processes described below, including the processes in FIGS. 5A-5B.

FIG. 2A illustrates electronic device 200 (e.g., portable multifunction device 100 or a device having one or more features of device 100). In the exemplary example of FIGS. 2A-2J, device 200 is a smartphone (e.g., a smartphone with an Android® or iOS® operating system). In other examples, electronic device 200 can be a different type of electronic device, such as a laptop, desktop, or tablet computer. Electronic device 200 includes touchscreen 202, which can receive inputs in the form of user contacts.

In FIG. 2A, device 200 displays home interface 204, which is a home screen of a mobile application programmed to delivery targeted digital therapies to a user. In some embodiments, a digital therapy (also referred to as a “skill” or digital-therapeutic in the exemplary user interfaces) delivers an evidence-based psychosocial intervention/technique via a computing (e.g., mobile or web) interface. In some embodiments, the user is an individual diagnosed with schizophrenia and therefore in need of therapies for addressing one or more symptoms of schizophrenia. In some embodiments, interface 204 is accessible (e.g., only accessible) after a user completes an enrollment process and/or is authenticated (e.g., via a passcode or biometric authentication technique, such as fingerprint or facial recognition). Interface 204 includes selectable icon 206, corresponding to a check-in screen, selectable icon 208, corresponding to a skill practice screen, variable information region 210 (e.g., for displaying different text content), and selectable icon 212, corresponding to a variable, specific skill (e.g., a specific skill such as that shown in FIG. 2E and discussed below). Interface 204 also includes a selectable icon region 214 that includes selectable icons for accessing various interfaces of the mobile application, as discussed in more detail below. While displaying interface 204, device 200 receives (e.g., detects) input 216 corresponding to selectable icon 206.

At FIG. 2B, in response to input 216, device 200 displays check-in interface 218, which is a check-in or query screen of the mobile application. As seen in FIG. 2B, interface 218 includes more content than can be displayed at one time on touchscreen 202. A user can access the additional content by scrolling (e.g., via a swipe gesture) interface 218. In some embodiments, check-in interface 218 is accessed by selecting selectable icon 220 in selectable icon region 214, which is persistent through most or all of the interfaces of the mobile application. As seen in FIG. 2B, selectable icon 220, corresponding to check-in interface 218, is emphasized to indicate that the check-in screen is currently displayed. Interface 218 includes a plurality of queries 222, including first query 222a (“Have you talked to other people recently?”) and second query 222b (“Do you feel in control of your voices right now?”). Each query of interface 218 corresponds to a subset of one or more skills of a larger set of available skills. For example, first query 222a corresponds to skills for developing confidence in social situations while second query 222b corresponds to skills for improving control over voices (e.g., auditory hallucinations). Each query is associated with a graphical icon that also corresponds to the same subset of one or more skills as the query, providing users with a sense of continuity between the query and corresponding subset of skills. Each query is also associated with a set of positive and negative response selectable icons for providing a positive or negative response to the corresponding query. Interface 218 also includes a selectable completion icon 224 for submitting provided responses to the plurality of queries. In some embodiments, completion icon 224 is conditional selectable and only becomes active upon selection of a response to at least one query of the plurality of queries. As shown in FIG. 2B, device 200 has already received inputs corresponding to responses to first query 222a (a negative response) and second query 22b (a positive response). In some embodiments, a user can change the response to a given query freely, prior to selecting completion icon 224. In some embodiments, the state of selected responses is persistent for at least period of time (e.g., an hour, a day). In some embodiments, the state of selected responses resets (e.g., reverts to a cleared and empty state) at a predetermined time (e.g., at midnight), each day.

At FIG. 2C, device 200 displays a results interface 226, which is a check-in results screen of the mobile application. Interface 226 includes a first region 228 that summarizes queries to which a positive response was provided on interface 218. Interface 226 also include a second region 230 that summarizes queries to which a negative response was provided. As shown in FIG. 2C, device 200 has received responses for all 10 of the plurality of queries shown in check-in interface 218. In some embodiments, interface 226 also includes a third region summarizing queries to which no response was provided. Interface 226 also includes a selectable icon 232 for returning to interface 218 and providing new or revised responses to one or more the plurality of queries 222. Second region 230 includes a selectable icon corresponding to each of the queries for which a negative response was received, including selectable icon 230a, corresponding to second query 222b of interface 218. Each of the selectable icons of second region 230 provides access to a corresponding subset of skills. While displaying interface 226, device receives input 234 corresponding to selectable icon 230a.

At FIG. 2D, in response to receiving input 234, device 200 displays skill subset interface 236, which is a skills screen specific to a subset of skills for improving control over voices. Interface 236 includes a plurality of selectable skill icons 238, including skill icons 238a (“Tune out your voices”) and 238b (“Voices are all bark and no bite”), each directed to therapeutically assisting users experiencing auditory hallucinations in the form of hallucinatory voices. Interface 236 also includes selectable icon 240 for accessing a general skill interface, discussed in more detail below. As shown in FIG. 2D, skill 238a includes a single star icon and skill 238b includes two star icon. In some embodiments, each star icon represents a predetermined level of mastery of the respective skill. In some embodiments, a level of mastery can indicate a number of times that the specific skill has been completed (e.g., 1 star indicates a single completion, 2 stars represents two completions, and so forth). In some embodiments, a single level of mastery corresponds to multiple completions (e.g., 5 completions for every level of mastery, as indicated by a single star). While displaying interface 236, device 200 receives (e.g., detects) input 242 corresponding to selectable skill icon 238a.

At FIG. 2E, in response to receiving input 242, device 200 displays specific skill interface 244, which is a user interface for delivery of a specific therapeutic skill. Specifically, user interface 244 is an interface for delivering a skill (e.g., a psychosocial intervention) that assists a user experiencing auditory hallucinations (e.g., in the form of voices in the user's head). Interface 244 includes a multimedia region 246 and a text region 248a. In some embodiments, device 200 plays, upon receiving an input corresponding to region 246, a video guiding the user through one or more evidence-based exercises for coping with auditory hallucinations. In some embodiments, the video includes at least 4 parts: normalization, steps for successful skill practice, explanation of how this skill can help, and encouragement to practice the skill immediately. In some embodiments, one or more skills are action-oriented, and can be completed quickly (e.g., in less than 5 minutes), and are designed to be practiced multiple times. Text region 248a initially displays scrollable text guiding the user through the exercises for coping with auditory hallucinations. Interface 244 also includes a selectable icon 250 for completing the skill corresponding to interface 244 and selectable icon 252 for displaying informational text corresponding to the specific skill of user interface 244 (i.e., a skill for coping with auditory hallucinations). In some embodiments, selectable icon 250 for completing the skill is only selectable once the video has been completely played and/or the text in region 248a has been scrolled to the end and the skill/digital therapy is considered delivered only after the video has been completely played and/or the text in region 248a has been scrolled. In the embodiment shown in FIG. 2E, skill interface screen 244 also includes a star icon 244a, indicating that the user has a first level of mastery of the skill corresponding to interface 244. While displaying interface 244, device 200 detects input 254 corresponding to selectable icon 252.

At FIG. 2F, in response to input 254, text region 248a is replaced with text region 248b, which displays informational text corresponding to the specific skill of user interface 244. While displaying user interface 244 with text region 248b and while icon 250 is selectable, device 200 detects input 256 corresponding to selectable icon 250.

At FIG. 2G, in response to input 256, device 200 displays first skill delivery completion interface 258, which indicates that the user has achieved a second level of mastery of the specific skill of user interface 244. In some embodiments, device 200 displays interface 258 in response to an input corresponding to selectable icon 250 while displaying interface 244 with text region 248a. Interface 258 includes selectable next icon 260 for progressing to the next interface. While displaying interface 258, device 200 detects input 262 corresponding to icon 260.

At FIG. 2H, in response to input 262, device 200 displays a second skill delivery completion interface 264. Interface 264 includes selectable icon 266 for adding the specific skill of interface 244 to a toolbox (e.g., a repository) of user-selected skills of the larger set of available skills. Interface 264 also includes selectable icon 268 to decline to add the specific skill of interface 244 to the toolbox of user-selected skills. In some embodiments, second skill delivery completion interface 264 also includes a selectable icon for exploring additional skills of the larger set of available skills (e.g., exploring a skill that also relates to the same category of skills as the specific skill of interface 244). In some embodiments, if the specific skill is already included in the toolbox, selectable icons 266 and 268 are not displayed as part of interface 264; rather, interface 264 includes a selectable icon for viewing the specific skill in a user interface (e.g., interface 270 discussed below) corresponding to the toolbox and/or a selectable icon for returning to the home interface 204.

In some embodiments, device 200 displays (e.g., as part of interface 264 or in a third skill delivery completion interface) a selectable icon that, when selected, causes display of general skill interface 274 (discussed below). In some embodiments, device 200 displays (e.g., as part of interface 264 or in a third skill delivery completion interface) a selectable icon that, when selected, causes display of check-in interface 218 (discussed above). In some embodiments, display of these selectable icons is conditional. For example, display of the selectable icon that, when selected, causes display of check-in interface 218, occurs if the skill delivery skill completion interface(s) were displayed after completing a skill that was arrived at via the check-in interface 218. Similarly, display of the selectable icon that, when selected, causes display of general skill interface 274, occurs if the skill delivery skill completion interface(s) were displayed after completing a skill that was arrived at via the general skill interface 274.

At FIG. 2I, device 200 displays toolbox interface 270. In some embodiments, device 200 displays interface 270 in response to an input corresponding to the toolbox in selectable icon region 214 (e.g., while displaying the home interface 204 and/or check-in interface 218). In some embodiments, device 200 displays interface 270 in response to an input received while displaying second skill delivery completion interface 264. Toolbox interface 270 includes plurality of user-selected skills 272, including a first selectable icon 272a corresponding to a first specific skill (“Tune out your voices”) and a second selectable icon 272b corresponding to a second specific skill (“Voices are all bark and no bite”). In some embodiments, device 200 displays, in response to an input corresponding to selectable icon 272a, specific skill interface 244 which corresponds to the “Tune out your voices” skill.

At FIG. 2J, device 200 displays a general skill interface 274 for accessing the entire set of skills available in the mobile application. In some embodiments, device 200 displays interface 274 in response to an input corresponding to the skills icon in selectable icon region 214 (e.g., while displaying the home interface 204 and/or check-in interface 218). Interface 274 includes a plurality of selectable skill category icons 276. Skill category icons 276 includes a first selectable skill category icon 276a (“Have more control over voices”) that provides access to skill subset interface 236, discussed above, which can also be accessed via second region 230 of results interface 226. Skill category icons 276 also includes second selectable skill category icon 276b for accessing a different skill subset for skills that assist a user with being confident in social situations. In some embodiments, each skill category of skill category icons 276 corresponds to a query of the plurality of queries 222 of check-in interface 218.

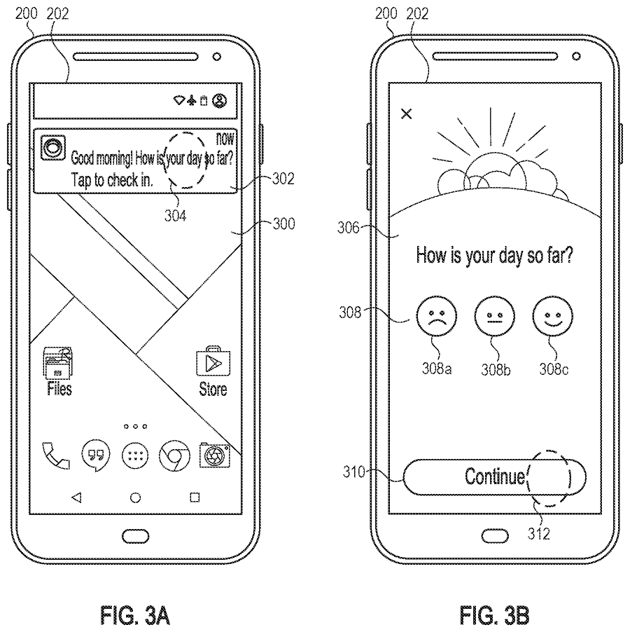

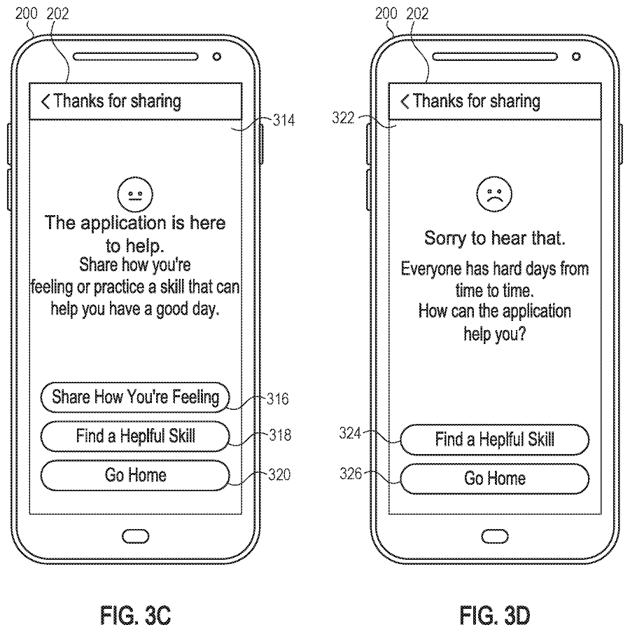

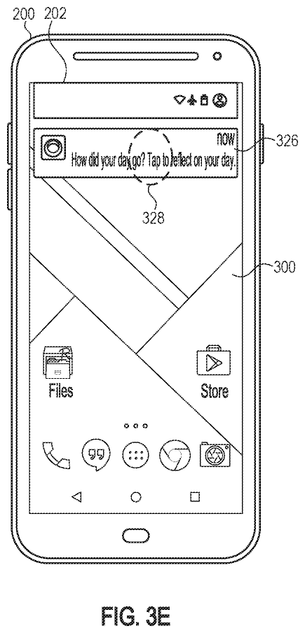

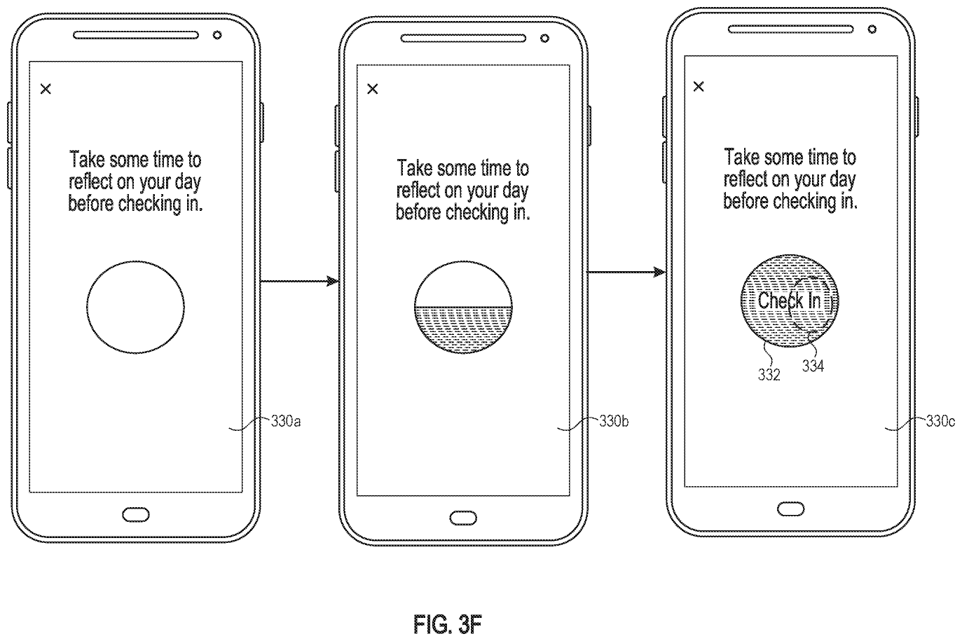

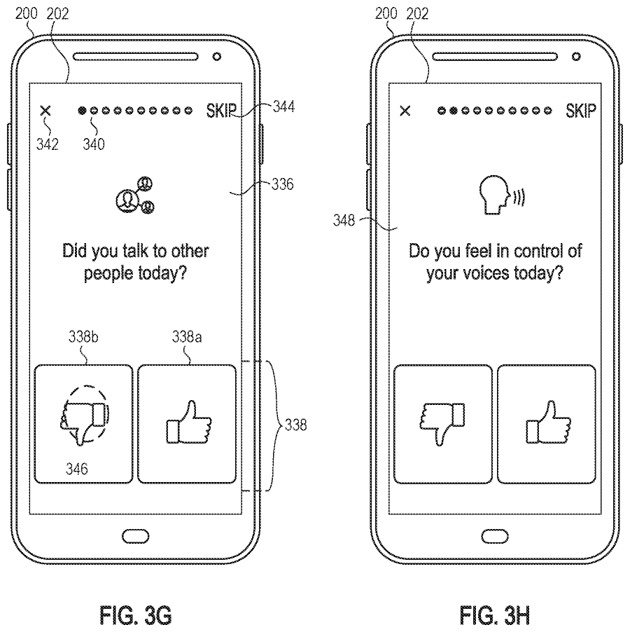

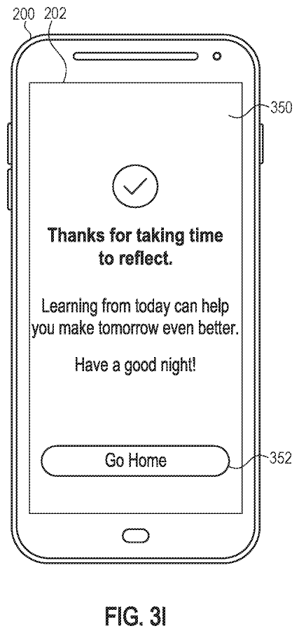

FIGS. 3A-3I illustrate exemplary user interfaces for notifications associated with the mobile application for providing targeted delivery of digital therapies. In some embodiments, the mobile applications generates three different types of notifications, on a daily basis (e.g., at 11 AM, 4 PM, and 9 PM or 11 AM, 2 PM, 6 PM, respectively). Two of the types of notification are discussed in more detail, below.

FIG. 3A illustrates electronic device 200 displaying a first notification 302 (e.g., a first type of notification) while displaying an operating screen home screen 300. In some embodiments, notifications of the first type are displayed in the morning of each day. While displaying notification 302, device 200 receives input 304 corresponding to notification 302.

At FIG. 3B, in response to input 304, device 200 displays daily check-in interface 306. Interface 306 includes plurality of selectable check-in icons 308 that include positive (icon 308a), negative (icon 308b), and nonspecific (icon 308c, also referred to as a “meh” response or “neutral” response) responses. Interface 306 also includes selectable completion icon 310 for submitting a provided response. In some embodiments, completion icon 310 is conditional selectable and only becomes active upon selection of a response. In some embodiments, a user can change the response freely, prior to selecting completion icon 310. While displaying interface 306 and after receiving an input on icon 308c, device 200 detects input 312 on completion icon 310.

At FIG. 3C, in response to receiving input 312 while the response corresponding to icon 308c is selected, device 200 displays nonspecific response interface 314. Interface 314 includes a selectable icon 316 (“Share How You're Feeling”) and a selectable icon 318 (“Find a Helpful Skill”). In some embodiments, upon receiving an input corresponding to icon 316, device 200 displays check-in interface 218 (FIG. 2B). In some embodiments, upon receiving an input corresponding to icon 318, device 200 displays general skill interface 274 (FIG. 2J). Interface 314 also includes selectable icon 320 for accessing home interface 204 (FIG. 2A).

If, while displaying check-in interface 306, device 200 receives a series of inputs (including selection of negative response icon 308b) corresponding to submission of a negative response, device 200 displays negative response interface 322 of FIG. 3D. Interface 322 includes a selectable icon 324 that, upon selection, causes device 200 to display general skill interface 274 (FIG. 2J). Interface 322 also includes selectable icon 326 for accessing home interface 204 (FIG. 2A).

If, while displaying check-in interface 306, device 200 receives a series of inputs (including selection of positive response icon 308a) corresponding to submission of a positive response, device 200 displays a positive response interface. In some embodiments, the positive response interface includes a selectable icon for accessing home interface 204 (FIG. 2A).

FIG. 3E illustrates electronic device 200 displaying a second notification 326 (e.g., a second type of notification) while displaying an operating screen home screen 300. In some embodiments, notifications of the second type are displayed in the evenings of each day. While displaying notification 326, device 200 receives input 328 corresponding to notification 326.

At FIG. 3F, in response to input 328, device 200 displays a plurality of daily reflection interfaces 330, including interfaces 330a (initial), 330b (intermediate), and 330c (terminal) which depict an animated sequence that occurs over a predetermined time period (e.g., 15 seconds, 30 seconds, 60 seconds). In some embodiments, the duration of the predetermined time period is selected to provide the user with time to reflect upon the course of the user's day. Upon completion of the animated sequence (and expiration of the predetermined time period) icon 332 becomes selectable. While icon 332 is selectable, device 200 detects input 334 corresponding to icon 322.

At FIG. 3G, in response to input 334, device 200 displays first reflection query interface 336 corresponding to a first reflection query (“Did you talk to other people today?”) of a plurality of reflection queries. Interface 336 includes selectable response icons 338, including positive response icon 338a and negative response icon 338b. Interface 336 also includes indicators 340 that indicate the position of the current reflection query in the plurality of total reflection queries. Interface 336 also includes selectable exit icon 342 for exiting the query sequence and accessing home interface 204 (FIG. 2A) and skip icon 344 for progressing to the next reflection query without providing a response to the first reflection query. While displaying interface 336, device 200 receives input 346, corresponding to positive response icon 338a.

At FIG. 3H, in response to input 346, device 200 displays second reflection query interface 348 corresponding to a second reflection query (“Did you feel in control of your voices today?”). Second reflection query interface 348 includes user interface elements similar to those discussed with respect to interface 336.

FIG. 3I depicts a reflection query completion interface 350 that is displayed by device 200 after the user has progressed through the plurality of reflection queries (e.g., by providing responses or selection of skip icons). Interface 350 includes selectable icon 352 for accessing home interface 204 (FIG. 2A).

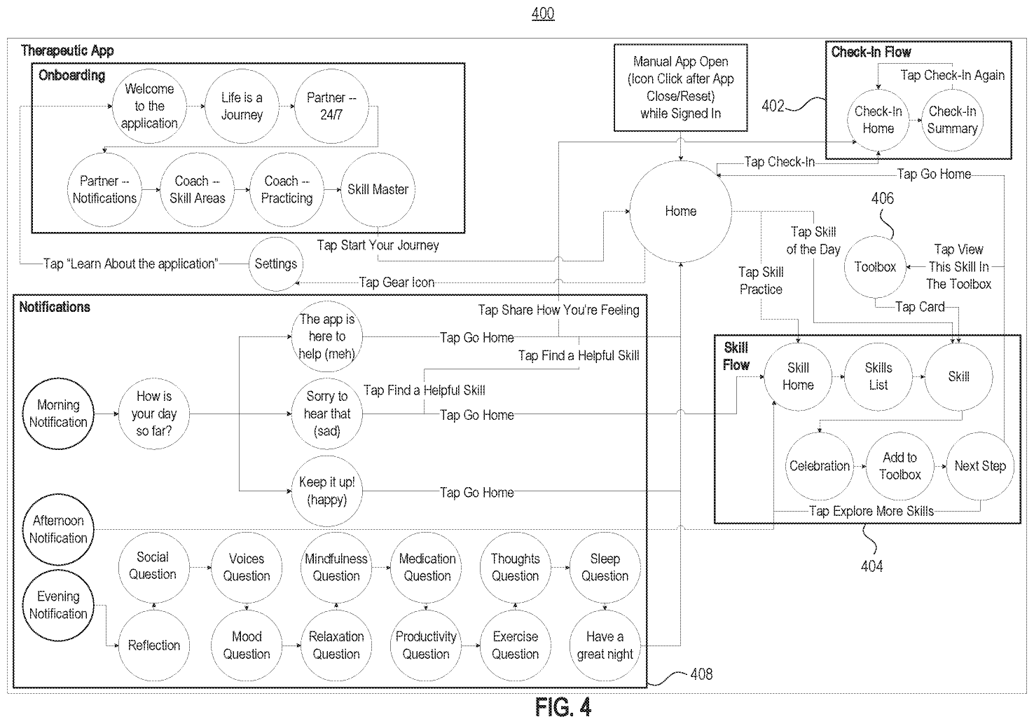

FIG. 4 illustrates an exemplary state diagram 400 showing the relationships and progressions between the various user interfaces of FIGS. 2A-2J and 3A-3I. State diagram 400 includes check-in states 402 corresponding to check-in interface states (e.g., interfaces 218 and 226). State diagram 400 also includes skill flow states 404 corresponding to skill-related interface states (e.g., interfaces 236, 244, 258, 264, and 274). State diagram 400 further includes toolbox state 406 corresponding to a tool interface state (e.g., interface 21). State diagram 400 also includes notification states 408 corresponding to notification-related interface states (e.g., interfaces 302, 306, 314, 322, 326, 330a-330c, 344, 348, and 350).

FIGS. 5A-5B are flow diagrams illustrating a method for providing targeted delivery of digital therapies, in accordance with some examples, including the examples of FIGS. 2A-2J and 3A-3I. Method 500 is performed at an electronic device (e.g., device 100 or 200) with a display and a input device (e.g., a touchscreen). Some operations in method 500 are, optionally, combined, the orders of some operations are, optionally, changed, and some operations are, optionally, omitted.

As described below, method 500 provides an intuitive way for providing targeted delivery of digital therapies. The method reduces the cognitive burden on a user for accessing digital therapies, thereby creating a more efficient human-machine interface. For battery-operated computing devices, enabling a user to access digital therapies faster and more efficiently conserves power and increases the time between battery charges.

The electronic device (e.g., 100, 200) displays (502), on the display, a query interface (e.g., 218), the query interface presenting a plurality of queries (e.g., 222), including a first query (e.g., 222b).

In some embodiments, the first query is associated with a first symptom of schizophrenia and the first set of digital therapies is configured to address the first symptom of schizophrenia. In some embodiments, the first query is associated with a first aspect of treatment of schizophrenia (e.g., auditory hallucinations) and the first set of digital therapies are associated with improving the first aspect of treatment of schizophrenia (e.g., improving voice control).

While displaying the query interface, the electronic device receives (504), via the input device (e.g., touchscreen 202), a first sequence of inputs (e.g., selection (e.g., a tap on a response option) of responses to one or more of the queries and selection of finish icon 224), including an input corresponding to a response to at least the first query (e.g., “Do you feel in control of your voices right now?”).

In response to receiving the first sequence of inputs (In some embodiments, in response to the first sequence of inputs, the electronic device determines (506) whether the response(s) are of a first type or a second type), the electronic device displays (508) a response summary interface (e.g., 226).

In accordance with the response to the first query being a response of a first type (e.g., a negative response), the electronic device displays (510), in a first region (e.g., a lower region; 230) of the response summary interface, a first response element (e.g., 230a) corresponding to the first query, the first response element including at least a first selectable graphical element (e.g., a selectable icon for accessing a specific set of therapeutic skills) corresponding to a first set of digital therapies (e.g., a subset of a larger set of available digital therapies (e.g., digital-therapeutics), the first set specifically relating to the first query (e.g., a set of digital therapies corresponding to stress and anxiety therapies) (e.g., the skills of interface 236) associated with the first query.

In some embodiments, the electronic device while displaying the first response element (e.g., 230a), receives a second sequence of inputs, including an input corresponding to selection of the first selectable element. In response to receiving the second sequence of inputs, the electronic device displays a first set of selectable graphical elements corresponding to the first set of digital therapies (e.g., displaying a first set of selectable icons associated with the first set of digital therapies “Have more control over voices”); displaying interface 236), including at least a third selectable graphical element corresponding to a first digital therapy associated with (e.g., related to the context of) the first query. While displaying the first set of digital therapies (e.g., 238), the electronic device receives a third sequence of inputs, including an input corresponding to selection of the third selectable graphical element (e.g., 238a) corresponding to the first digital therapy. In response to receiving the third sequence of inputs, the electronic device outputs the first digital therapy (e.g., interface 244), the first digital therapy including at least a multimedia component (e.g., a video or audio component; e.g., 246).

In some embodiments, after outputting the first digital therapy, the electronic device displays an option (e.g., 266) to categorize (e.g., add or flag) the first digital therapy as a designated digital therapy (e.g., a digital therapy that is accessible in a toolbox (e.g., a repository of frequently used or preferred therapies)). The electronic device receives an input corresponding to a selection of the option (e.g., 266) to categorize the first digital therapy as a designated digital therapy. In response to receiving the input corresponding to the selection of the option to categorize the first digital therapy as a designated digital therapy, categorizing the first digital therapy as a designated digital therapy (e.g., adding the first digital therapy to the toolbox corresponding to interface 270).

In some embodiments, after categorizing the first digital therapy as a designated digital therapy, the electronic device receives a request (e.g., selection of toolbox icon from the home interface 204; selection of a “view this skill in the toolbox” option after completing a skill and adding it to the toolbox) to display a designated digital therapy interface (e.g., a toolbox interface 270). In response to the request to display the designated digital therapy interface, the electronic device displays the designated digital therapy interface (e.g., 270), the designated digital therapy interface including at least a fourth selectable graphical element (e.g., 272a) corresponding to the designated first digital therapy (e.g., including a selectable icon for accessing the first digital therapy). (In some embodiments, designated digital therapies are displayed with an indication (e.g., one or more stars) of the number of times the digital therapy has been accessed/outputted.).

In accordance with the response to the first query being a response of a second type (e.g., a positive response), different than the first type, the electronic device displays (512), in a second region (e.g., a region distinct from the first region, an upper region; 228) of the response summary interface, a second response element (e.g., a synopsis of the matching query(s); acknowledgement text), wherein the second response element corresponds (e.g., is associated with and/or based on the first query) to the first query and does not include (e.g., the second response element does not provide access to corresponding digital therapies) the first selectable graphical element corresponding to the first set of digital therapies associated with the first query (In some embodiments, the second response element includes the first selectable graphical element).

In some embodiments, the plurality of queries (e.g., 222) includes a second query (e.g., a query different than the first query; 222a) and the first sequence of inputs includes a response to the second query (514) (e.g., “Have you talked to other people recently?”). The electronic device, further in response to the first sequence of inputs and in accordance with the response to the second query being a response of the first type (e.g., an negative response to the second query), displays (516), in the first region (e.g., 230) of the response summary interface, a third response element (e.g., acknowledgement text based on the specifically provided response to the second query) corresponding to the second query, the third response element including at least a second selectable graphical element corresponding to a second set of digital therapies (e.g., different from the first set of digital therapies, a second subset of the larger set of digital therapies, the second set specifically relating to the second query (e.g., a set of digital therapies corresponding to social situation therapies)) associated with the second query. The electronic device, further in response to the first sequence of inputs and in accordance with the response to the second query being a response of the second type (e.g., a positive response to the second query), displays (518), in the second region (e.g., 228) of the response summary interface, a fourth response element, wherein the fourth response element corresponds (e.g., is associated with and/or based on the first query) to the second query and does not include (e.g., the second response element does not provide access to corresponding digital therapies) the second selectable graphical element corresponding to the second set of digital therapies associated with the second query.

In some embodiments, prior to displaying the query interface, the electronic device displays a home interface (e.g., 204), the home interface including a second set of selectable graphical elements, the second set of selectable graphical elements including at least a selectable graphical element (e.g., 206) configured to cause display of the query interface (In some embodiments, the home interface further includes selectable graphical elements that cause display of the toolbox and the digital therapy interface, respectively). While displaying the home interface, the electronic device receives an input (e.g., 216) corresponding to selection of the selectable graphical element configured to cause display of the query interface. Displaying the query interface (e.g., 218) occurs in response to receiving the input (e.g., 216) corresponding to selection of the selectable graphical element (e.g., 206) configured to cause display of the query interface.

In some embodiments, the home interface further includes a selectable graphical element (e.g., “skills” icon of region 214) configured to cause display of a digital therapies interface (e.g., 274). The electronic device receives an input corresponding to selection of the selectable graphical element configured to cause display of the digital therapies interface. In response to receiving the input corresponding to selection of the selectable graphical element configured to cause display of the digital therapies interface, the electronic device displays the digital therapies interface (e.g., a screen of a plurality (e.g., all) of digital therapy categories available on the electronic device), wherein the digital therapies interface includes at least a fifth selectable graphical element (276a) corresponding to the first set of digital therapies (e.g., a set of digital therapies corresponding to voice control therapies); and a sixth selectable graphical element (276b) corresponding to the second set of digital therapies (e.g., a set of digital therapies corresponding to social situation therapies).

In some embodiments, the electronic device determines whether a plurality of sets of criteria (e.g., sets of criteria for outputting morning, afternoon, and evening notifications, respectively) are met. In accordance with a determination that a first set of criteria (e.g., a time-based criteria corresponding to the morning) of the plurality of sets of criteria are met, the electronic device outputs a notification of a first type (e.g., a morning notification; notification 302). In accordance with a determination that a second set of criteria (e.g., a time-based criteria corresponding to the evening) of the plurality of sets of criteria are met, the second set of criteria being different than the first set of criteria, the electronic device outputs a notification of a second type (e.g., an evening notification; notification 326), different than the first type.

In some embodiments, while the notification of the first type (e.g., 302) is displayed, the electronic device receives an input (e.g., 304) corresponding to selection of the notification of the first type. In response to receiving the input corresponding to selection of the notification of the first type; displaying a third query (e.g., “how is your day?”) and a third set of selectable graphical elements (e.g., 308) corresponding to a plurality of responses to the third query (e.g., a positive response, a negative response, a nonspecific or noncommittal response). The electronic device receives a fourth sequence of inputs (e.g., a selection of a response and a selection of a “continue” option 312), including an input corresponding to selection of a selectable graphical element of the third set of selectable graphical elements. In response to receiving the fourth sequence of inputs and in accordance with the fourth sequence of inputs including a selection of a first selectable graphical element (e.g., a graphical element corresponding to a nonspecific or noncommittal response (e.g., a response that is not fully negative or fully positive) of the third set of selectable graphical elements (e.g., 308c), the electronic device displays a first response interface (e.g., 314) that includes a selectable graphical element (e.g., 316) configured to cause display of the query interface and a selectable graphical element (e.g., 318) configured to cause display of the digital therapies interface. In response to receiving the fourth sequence of inputs and in accordance with the fourth sequence of inputs including a selection of a second selectable graphical element (e.g., a graphical element corresponding to a negative response (e.g., 308a)) of the third set of selectable graphical elements, displaying a second response interface (e.g., 322) that includes a selectable graphical element (e.g., 324) configured to cause display of the digital therapies interface. In some embodiments, the second response interface does not include a selectable graphical element configured to cause display of the query interface. In some embodiments, in accordance with the fourth sequence of inputs including a selection of a third selectable graphical element (e.g., a graphical element corresponding to a positive response) of the third set of selectable graphical elements, displaying a third response interface that does not include a selectable graphical element configured to cause display of the digital therapies interface and does not include a selectable graphical element configured to cause display of the query interface.

In some embodiments, while the notification of the second type (e.g., 326) is displayed, the electronic device receives an input (e.g., 328) corresponding to selection of the notification of the second type. In response to receiving the input corresponding to selection of the notification of the second type, the electronic device displays a first interstitial interface (e.g., an interface prompting the user to reflect on the day for a predetermined period of time; 330a and/or 330b). After displaying the first interstitial interface for a predetermined period of time (e.g., 15, 30, 60 seconds), the electronic device transitions (e.g., replacing the first interstitial interface or modifying the first interstitial interface) the first interstitial interface to a second interstitial interface (e.g., 330c), the second interstitial interface includes a selectable graphical element (332) for causing display of a fourth query (e.g., a query different than the third query). While displaying the second interstitial interface, the electronic device receives an input (e.g., 334) corresponding to selection of the selectable graphical element for causing display of the fourth query (e.g., a question relating to an evening check-in (e.g., “did you talk to other people today?”)). In response to receiving the input corresponding to selection of the selectable graphical element for causing display of a fourth query, the electronic device displays the fourth query (e.g., displays interface 336).

### Example 2

### Clinical Testing

| Study type | Interventional |
|---|---|
| Purpose and | The purpose of the study is to determine in patients currently |
| rationale | being administered antipsychotic pharmacotherapy whether the |
| | digital-therapeutics application can further reduce symptoms of |
| | schizophrenia as measured by the Positive and Negative |
| | Syndrome Scale (PANSS). |
| | The overall rationale for the study is to assess the first |
| | prescription digital therapeutic (PDT) in schizophrenia using a |
| | form of proven psychosocial intervention, cognitive behavioral |
| | therapy (CBT), to supplement standard of care with antipsychotic |
| | medications. |
| Study design | This is a randomized, sham controlled, rater-blinded, parallel |
| | group trial. Patients will be randomized 1:1 to receive the digital- |
| | therapeutics application or a sham control. The study includes an |
| | up to 4-week screening period, a 12-week treatment period, and |
| | a 4-week follow-up period. |
| Population | 102 male and female adults (18-65 years of age, inclusive) with |
| | a diagnosis of schizophrenia |
| Key inclusion | Signed informed consent must be obtained prior to |
| criteria | participation in the study. |
| | Healthy male and female subjects 18 to 65 years of age, |
| | inclusive, and in good health as determined by medical |
| | history, physical examination, and vital signs at screening |
| | SCID-based Diagnostic and Statistical Manual of Mental |
| | Disorders, Fifth Edition (DSM-5) diagnosis of schizophrenia |
| | and a total PANSS score >60 |
| | Proficient in English at 5th grade reading level or higher, in |
| | the judgement of the investigator |
| | Capable of using a mobile device (compatible with the |
| | digital-therapeutics application) and using common |
| | applications, in the judgement of the investigator |
| Key exclusion | Major change in primary antipsychotic medication in the |
| criteria | prior 4 weeks before screening (e.g., switching to a new |
| | agent or a major dose adjustment within two weeks of |
| | randomization) |
| | Planning to move out of the geographic area within 3 |
| | months |
| | Unable to use English to participate in the consent process, |
| | the interventions or assessments |
| | Inability to comply with study procedures, due to severe |
| | medical conditions or otherwise |
| | Meet DSM-5 diagnosis for a current episode of major |
| | depression, mania, or hypomania in the past month |
| | Meet DSM-5 diagnosis for a current moderate or severe |
| | alcohol or cannabis use disorder in the past 2 months |
| | Meet DSM-5 diagnosis for a current substance use disorder |
| | (other than alcohol or cannabis) in the past 2 months |
| | Considered high risk for suicidal behavior based on ISST- |
| | Plus score at screening, in the judgement of the investigator |
| Study treatment | the digital-therapeutics application |
| | Sham control |
| Efficacy | PANSS |
| assessments | BMQ |
| | MAP-SR |
| | WHOQOL-BREF |
| | BDI-II |
| Key safety | Adverse event monitoring |
| assessments | Vital signs |
| | ISST-Plus |

Objectives and Endpoints

Primary Objective

| Objective | Endpoint |
|---|---|
| To assess the efficacy of the digital- | Change in total PANSS score |
| therapeutics application as an adjunct | from baseline to day 85 or |
| to existing clinician-directed | last visit |
| pharmacotherapy to reduce symptoms | |
| of schizophrenia | |
| To evaluate retention to assigned study | Percent dropout rate |
| treatment | |

Secondary Obiectives

| Objectives | Endpoints |
|---|---|
| To evaluate the efficacy of the digital- | Change in the Positive PANSS |
| therapeutics application as an adjunct to | score from baseline to day 29, day |
| existing clinician-directed | 57, and day 85 or last visit |
| pharmacotherapy to reduce positive | Change in the General |
| symptoms of schizophrenia | Psychopathology PANSS score |
| | from baseline to day 29, day 57 and |
| | day 85 or last visit |
| To evaluate the efficacy of the digital- | Change in the Negative PANSS |
| therapeutics application as an adjunct to | score from baseline to day 29, day |
| existing clinician-directed | 57, and day 85 or last visit |
| pharmacotherapy to reduce negative | Change in the Motivation and |
| symptoms of schizophrenia | Pleasure self-report (MAP-SR) |
| | score from baseline to day 29, day |
| | 57, and day 85 or last visit |
| To assess safety and tolerability of the | Adverse events, serious adverse |
| digital-therapeutics application | events, and adverse events leading |
| | to discontinuation throughout the |
| | study |
| | Vital signs at baseline, day 85 or |
| | last visit |
| | InterSePT Scale for Suicidal |
| | Thinking-Plus (ISST-Plus) score at |
| | baseline, day 29, day 57, day 85, |
| | and day 115 or last visit |
| To evaluate the efficacy of the digital- | Change on the World Health |
| therapeutics application as an adjunct to | Organization Quality of Life |
| existing clinician-directed | (WHOQOL-BREF) scale from |
| pharmacotherapy to improve | baseline to day 29, day 57, and day |
| psychosocial functioning in patients with | 85 or last visit |
| schizophrenia | |
| To evaluate the efficacy of the digital- | Change in the Beck Depression |
| therapeutics application as an adjunct to | Inventory, Second Edition (BDI-II) |
| existing clinician-directed | total score from baseline to day 29, |
| pharmacotherapy to reduce depression | day 57, and day 85 or last visit |
| symptoms in patients with schizophrenia | |
| To evaluate the magnitude of the effect | Percentage change in PANSS score |
| of the digital-therapeutics application as | (within assigned group) from |
| an adjunct to existing clinician-directed | baseline to day 29, day 57, and day |
| pharmacotherapy to reduce symptoms | 85 or last visit |
| of schizophrenia | |
| To evaluate the response to treatment | Proportion of responders, defined as |
| | a reduction of at least 20% at day |
| | 85 or last visit in total PANSS score |
| | relative to baseline |
| To evaluate patient adherence to | Change in antipsychotic |
| antipsychotic medication | pharmacotherapy use as measured |
| | by the Brief Medication |
| | Questionnaire (BMQ) at day 29, day |
| | 57, and day 85 or last visit |

Exploratory Objectives

| Objectives | Endpoints |
|---|---|
| To evaluate the efficacy of the | Change on the Insomnia Severity |
| digital-therapeutics application | Index (ISI) from baseline to day 29, |
| as an adjunct to existing | day 57, and day 85 or last visit |
| clinician-directed pharmacotherapy | |
| to reduce sleep problems in | |
| patients with schizophrenia | |
| To evaluate retention to | Percent dropout rate in the digital- |
| assigned study treatment | therapeutics application and the |
| under different pharmacotherapy | sham group, by drug-class |
| conditions | |

Treatment Compliance

Treatment compliance here refers to subject use of the digital-therapeutics application. Data will be collected to assess frequency of subject use of the digital-therapeutics application, including whether use was prompted by a notification or subject-initiated.

Self-reported compliance with the subject's ongoing clinician-directed pharmacotherapy will additionally be assessed via the BMQ.

Measures of user engagement derived from app (such as time spent in app, number of therapeutic interactions, and domain use) will be explored graphically and using descriptive statistics.

Analysis of the Primary Endpoint(s)

Definition of Primary Endpoint(s)

The primary efficacy endpoint in the study is change in total PANSS score from baseline to day 85 or last visit, analyzed using the FAS.

Statistical Model, Hypothesis, and Method of Analysis

The change from baseline in total PANSS score will be analyzed using the mixed-effects model for repeated measures (MMRM; e.g., in Mallinckrodt C. H. et al., 2001, Journal of Biopharmaceutical Statistics, 11:9-21), including the fixed, categorical effects of treatment, visit, and treatment-by-visit interaction, as well as the continuous, fixed covariates of baseline score, baseline score-by-visit interaction, and disease duration at baseline. An unstructured (UN) covariance structure will be used to model the within-patient errors. The Kenward-Roger (KR) method will be used to adjust the estimated covariance of the mean difference and the degrees of freedom.

The primary hypothesis to be tested is that mean change from baseline in PANSS is greater with the digital-therapeutics application than with sham at day 85 or last visit. The primary comparison will be the contrast between treatments on day 85. The significance test will be carried out using 1-sided a=0.05. In addition, two-sided 90% confidence intervals for the treatment mean differences, as well as individual treatment mean change from baseline values at each study visit will be constructed.

If the primary analysis with UN covariance structure fails to converge, other covariance structures, including but not limited to the heterogeneous Toeplitz, antedependence (ANTE(1)), first-order autoregressive (AR(1)), and heterogeneous AR(1), will be tested. The covariance structure converging to the best fit, as determined by Akaike's information criterion, will be used as the primary analysis.

Analysis of Secondary Endpoints

Efficacy Endpoint(s)

The secondary efficacy endpoints listed below will be analyzed separately using an MMRM analysis. The change from baseline to each post-baseline visit will be the dependent variable.

The model will include the fixed, categorical effects of treatment, visit, and treatment-by-visit interaction, as well as the continuous, fixed covariates of baseline score, baseline score-by-visit interaction, and disease duration at baseline (in years). The analysis strategy will be the same as for the primary outcome. The outcomes to be analyzed are:
change from baseline in the positive PANSS score;
change from baseline in the General Psychopathology PANSS score;
change from baseline in the negative PANSS score;
change from baseline in the MAP-SR score;
change from baseline in the WHOQOL-BREF scale;
change from baseline in the BDI-II total score

In addition, the percentage change from baseline in the total PANSS score will be analyzed using an MMRM approach. Estimates of mean percentage change from baseline to each study visit with (90% Cls) will be obtained for each treatment group.

The analysis of slope of the total PANSS score will be performed using an MMRM approach. The change from baseline score at each post-baseline visit will be the dependent variable. The model will include the fixed effects of treatment, time, treatment-by-time interaction, the baseline total PANSS score, baseline total PANSS score-by-time interaction, and disease duration at baseline. Time will be assumed to be a continuous variable calculated as number of days between baseline and each visit post-baseline during the treatment period. The actual visit dates will be used to calculate number of days (time). The null hypothesis is that the contrast of the digital-therapeutics application vs. sham equals zero. To test for linearity, a quadratic slope model will be fitted. The quadratic model will include the linear component of time (TIME) and a quadratic component of time (TIME*TIME), the linear component of time and treatment interaction (TIME*TREATMENT) and quadratic component of time and treatment interaction (TIME*TIME*TREATMENT). If the quadratic component of time (TIME*TIME) and the interaction effect of TIM E*TIME*TREATMENT are not significant, the data can be considered as linear and the linear slopes model will be considered appropriate.

1. A method for the treatment of schizophrenia, in a subject, in need thereof, comprising administering to said subject an effective amount of an antipsychotic, wherein
i) the antipsychotic is provided in combination with a computer-implemented mobile device; and
ii) the computer-implemented mobile device comprises:
a display;
an input device;
one or more processors; and
memory storing one or more programs configured to be executed by the one or more processors, the one or more programs including instructions for:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
in accordance with the response to the first query being a response of a first type, displaying, in a first region of the response summary interface, a first response element corresponding to the first query, the first response element including at least a first selectable graphical element corresponding to a first set of digital therapies associated with the first query; and
in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

2. A method for the treatment of positive symptoms of schizophrenia, in a subject, in need thereof, comprising administering to said subject an effective amount of an antipsychotic, wherein,
i) the antipsychotic is provided in combination with a computer-implemented mobile device; and
ii) the computer-implemented mobile device comprises:
a display;
an input device;
one or more processors; and
memory storing one or more programs configured to be executed by the one or more processors, the one or more programs including instructions for:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
in accordance with the response to the first query being a response of a first type, displaying, in a first region of the response summary interface, a first response element corresponding to the first query, the first response element including at least a first selectable graphical element corresponding to a first set of digital therapies associated with the first query; and
in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

3. A method for the treatment of negative symptoms of schizophrenia, in a subject, in need thereof, comprising administering to said subject an effective amount of an antipsychotic, wherein,
i) the antipsychotic is provided in combination with a computer-implemented mobile device; and
ii) the computer-implemented mobile device comprises:
a display;
an input device;
one or more processors; and
memory storing one or more programs configured to be executed by the one or more processors, the one or more programs including instructions for:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
in accordance with the response to the first query being a response of a first type, displaying, in a first region of the response summary interface, a first response element corresponding to the first query, the first response element including at least a first selectable graphical element corresponding to a first set of digital therapies associated with the first query; and
in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

4. A method for treatment of depression associated with schizophrenia, in a subject, in need thereof, comprising administering to said subject an effective amount of an antipsychotic, wherein,
i) the antipsychotic is provided in combination with a computer-implemented mobile device; and
ii) the computer-implemented mobile device comprises:
a display;
an input device;
one or more processors; and
memory storing one or more programs configured to be executed by the one or more processors, the one or more programs including instructions for:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
in accordance with the response to the first query being a response of a first type, displaying, in a first region of the response summary interface, a first response element corresponding to the first query, the first response element including at least a first selectable graphical element corresponding to a first set of digital therapies associated with the first query; and
in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

5. A method for treatment of insomnia associated with schizophrenia, in a subject, in need thereof, comprising administering to said subject an effective amount of an antipsychotic, wherein,
i) the antipsychotic is provided in combination with a computer-implemented mobile device; and
ii) the computer-implemented mobile device comprises:
a display;
an input device;
one or more processors; and
memory storing one or more programs configured to be executed by the one or more processors, the one or more programs including instructions for:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
in accordance with the response to the first query being a response of a first type, displaying, in a first region of the response summary interface, a first response element corresponding to the first query, the first response element including at least a first selectable graphical element corresponding to a first set of digital therapies associated with the first query; and
in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

6. The method according to any one of claims 1 to 5, wherein the computer-implemented mobile device delivers behavioral therapy that focuses on symptoms of psychosis, mood management, sleep hygiene, medication adherence or social skills.

7. The method according to claim 6, wherein the behavioral therapy is cognitive behavioral therapy for schizophrenia, that focuses on one or more: i) symptoms of psychosis, ii) mood management, iii) sleep hygiene, iv) medication adherence and v) social skills.

8. A method for the treatment of schizophrenia, in a subject, in need thereof, comprising administering to said subject an effective amount of an antipsychotic, wherein
i) the antipsychotic is provided in combination with a digital-therapeutic; and
ii) the digital-therapeutic delivers behavioral therapy.

9. The method according to claim 8, wherein the digital-therapeutic is implemented via a mobile device selected from the group comprising a smartphone, a laptop computer, a tablet computer, and a wearable computer.

10. A method for the treatment of schizophrenia, in a subject, in need thereof, comprising administering to said subject an effective amount of an antipsychotic, wherein the method is combined with computer-implemented behavioral therapy.

11. The method according to claim 10, wherein the computer-implemented behavioral therapy is provided by a computerized device selected from the group comprising a smartphone, a laptop computer, a tablet computer, and a wearable computer.

12. A combination comprising an antipsychotic and a computer-implemented mobile device;
wherein the computer-implemented mobile device comprises:
a display;
an input device;
one or more processors; and
memory storing one or more programs configured to be executed by the one or more processors, the one or more programs including instructions for:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
in accordance with the response to the first query being a response of a first type, displaying, in a first region of the response summary interface, a first response element corresponding to the first query, the first response element including at least a first selectable graphical element corresponding to a first set of digital therapies associated with the first query; and
in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

13. A combination comprising an antipsychotic and a digital-therapeutic, wherein the digital-therapeutic delivers behavioral therapy.

14. A computerized method for targeted delivery of digital therapies, the method comprising:
at an electronic device including a display and an input device:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
in accordance with the response to the first query being a response of a first type, displaying, in a first region of the response summary interface, a first response element corresponding to the first query, the first response element including at least a first selectable graphical element corresponding to a first set of digital therapies associated with the first query; and
in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

15. A non-transitory computer-readable storage medium storing one or more programs configured to be executed by one or more processors of an electronic device with a display and an input device, the one or more programs including instructions for:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
in accordance with the response to the first query being a response of a first type, displaying, in a first region of the response summary interface, a first response element corresponding to the first query, the first response element including at least a first selectable graphical element corresponding to a first set of digital therapies associated with the first query; and
in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.

16. An electronic device, comprising:
a display;
an input device;
one or more processors; and
memory storing one or more programs configured to be executed by the one or more processors, the one or more programs including instructions for:
displaying, on the display, a query interface, the query interface presenting a plurality of queries, including a first query;
while displaying the query interface, receiving, via the input device, a first sequence of inputs;
in response to receiving the first sequence of inputs, displaying a response summary interface, the response summary interface including:
in accordance with the response to the first query being a response of a first type, displaying, in a first region of the response summary interface, a first response element corresponding to the first query, the first response element including at least a first selectable graphical element corresponding to a first set of digital therapies associated with the first query; and
in accordance with the response to the first query being a response of a second type, different than the first type, displaying, in a second region of the response summary interface, a second response element, wherein the second response element corresponds to the first query and does not include the first selectable graphical element corresponding to the first set of digital therapies associated with the first query.
